(19) United States
(12) Patent Application Publication
(10) Pub. No.: US 2024/0125703 A1
(43) Pub. Date: Apr. 18, 2024

(54) SYSTEMS AND METHODS FOR QUANTIFYING USER OBSERVED VISUALIZATION OF FLUORESCENCE IMAGING AGENTS

(71) Applicants: Leticia DELGADO-HERRERA, Lake Forest, IL (US); Stryker Corporation, Kalamazoo, MI (US)

(72) Inventors: Paul Roald WESTWICK, Vancouver (CA); John Josef Paul FENGLER, North Vancouver (CA); Leticia DELGADO-HERRERA, Lake Forest, IL (US)

(21) Appl. No.: 18/468,671

(22) Filed: Sep. 15, 2023

Related U.S. Application Data
(60) Provisional application No. 63/376,065, filed on Sep. 16, 2022.

(51) Int. Cl. G01N 21/64; G01N 33/58; G06T 7/00

(52) U.S. Cl. CPC G01N 21/6428; G01N 33/582; G06T 7/0012; G01N 2021/6439; G06T 2207/30024

(57) ABSTRACT

A method for assessing fluorescence imaging agent-based visual enhancement in a color image in which a fluorescence imaging agent signal is represented by a first color, the method including: identifying at least one first region of the color image that is encompassed by a portion of anatomy or pathology of interest in which the fluorescence imaging agent is present; identifying at least one second region of the color image that is not encompassed by the anatomy or pathology of interest; and determining a measure of visual enhancement of the anatomy or pathology of interest in the color image by comparing a contribution of the first color relative to other colors in the at least one first region to a contribution of the first color relative to the other colors in the at least one second region.

### CROSS-REFERENCE TO RELATED APPLICATIONS

This application claims the benefit of U.S. Provisional Application No. 63/376,065, filed Sep. 16, 2022, the entire contents of which are hereby incorporated by reference herein.

### FIELD

This disclosure generally relates to fluorescence visualization, including image-guided surgery, and more particularly, to using a fluorescence imaging agent in conjunction with an imaging system for surgical visualization.

### BACKGROUND

Visual guidance using fluorescence imaging agents (contrast fluorescence imaging agent, contrast, contrast agent, and imaging agent are terms used interchangeable with fluorescence imaging agent) may be employed before, during, or after surgical procedures. For example, visual guidance using one or more fluorescence imaging agents may be employed during medical procedures, such as surgeries, to identify anatomically critical, but potentially difficult to see structures (e.g., nerves, blood vessels, ureters, etc.). Alternatively, such fluorescence imaging agents—either with or without a targeting ligand—may also be used to identify and localize pathology (e.g., cancers or other disease states).

The visualization of such fluorescence imaging agents can be by direct observation but may also utilize video technology—particularly in instances where the surgery is being performed minimally invasively or endoscopically (e.g., intraluminally) or where the visualization requires sensing wavelengths beyond the visible spectrum. Even for fluorescence imaging agents that are directly visible to the human eye, the visualization modality may benefit from the use of video technology (e.g., in the instance of fluorescence visualization where bright excitation light produces a fluorescence emission that is orders of magnitude dimmer). The video technology is typically used to induce, acquire and display the signal from such fluorescence imaging agents in real time during the surgery but is often also used to record the signal.

When using such fluorescence imaging agents in combination with video technology for image-guided surgery, the ease of visualization is a function of both the signal intensity and signal contrast of the fluorescence imaging agent relative to the surrounding tissues. In instances where the signal from the fluorescence imaging agent is being represented and displayed in a specific color by the video technology, the “color contrast” between tissues containing the fluorescence imaging agent and the surrounding tissues can provide an easily recognized—and clinically meaningful—visual cue for the surgeon.

A primary consideration in the use of such visualization fluorescence imaging agents relates to ensuring that sufficient fluorescence imaging agent is present in the tissue area of interest (e.g., in the critical anatomy or pathology). Simultaneously, ethical best practices mandate that not more fluorescence imaging agent is administered than what is required for the surgeon to visualize the tissue area of interest. In other words, in the interests of safety and efficacy, the amount of fluorescence imaging agent administered to human subjects should be both a sufficient and necessary dose. It is consequently desirable to near optimize or optimize the dose of administered fluorescence imaging agent to achieve this sufficient and necessary condition.

Furthermore, as multiple fluorescence imaging agents are developed for the purpose of similar image-guided surgery applications, it may be desirable to compare the ability of a given fluorescence imaging agent to highlight the critical anatomy or pathology with the performance of other fluorescence imaging agents.

Finally, it is recognized that—for the surgeon—the performance of a fluorescence imaging agent being utilized for image-guided surgery will also depend upon the nature and configuration of the optical imaging capture and display technology with which fluorescence imaging agent is being used. It is therefore potentially also desirable to have a means by which to quantitatively compare the performance of multiple types and configurations of optical imaging technology when used in conjunction with a given fluorescence imaging agent.

### SUMMARY

According to an aspect of this invention, methods that quantify the degree to which the representation of fluorescence imaging agent in a color image or surgical video contrasts with the colors of the surrounding tissues in the color image can be used to determine the near optimal or optimal dose of fluorescence imaging agent utilized for image-guided surgery, to quantify a surgeon's ability to visualize an anatomical feature, as a biomarker of a surgeon's ability to visualize an anatomical feature, to compare the performance of different fluorescence imaging agents, and/or to compare the performance of multiple types and configurations of visualization systems when used in conjunction with a given fluorescence imaging agent. The signal from a fluorescence imaging agent present in a tissue of interest may be represented in the color image or surgical video in a particular color (e.g., green, cyan, etc.) that may not be substantially present in the human body, and the signal from the surrounding tissues may be represented as they would appear under direct visualization with the human eye when illuminated by white light. The fluorescence imaging agent may have been administered prior to start of the method. The color image or surgical video may have been generated prior to start of the method. The signal from the fluorescence imaging agent and the signal from the surrounding tissues are used to quantify the color contrast between the tissue area of interest and the surrounding tissue. This color contrast may subsequently be used for determining the near optimal or optimal dose of fluorescence imaging agent utilized for image-guided surgery, for quantifying a surgeon's ability to visualize an anatomical feature, as a biomarker of a surgeon's ability to visualize an anatomical feature, for comparing the performance of different fluorescence imaging agents, and/or for comparing the performance of different visualization systems or visualization system settings.

According to an aspect, a method for assessing fluorescence imaging agent-based visual enhancement and/or conspicuity in a color image in which a fluorescence imaging agent signal is represented by a first color includes identifying at least one first region of the color image that is encompassed by a portion of anatomy or pathology of interest in which the fluorescence imaging agent is present; identifying at least one second region of the color image that is not encompassed by the anatomy or pathology of interest; and determining a measure of visual enhancement of the anatomy or pathology of interest in the color image by comparing a contribution of the first color relative to other colors in the at least one first region to a contribution of the first color relative to the other colors in the at least one second region. The fluorescence imaging agent may have been administered prior to start of the method. The color image or surgical video may have been generated prior to start of the method.

Optionally, the measure of visual enhancement is associated with a first dose of the fluorescence imaging agent, and the method further comprises comparing the measure of visual enhancement associated with the first dose with a measure of visual enhancement associated with a second dose of the fluorescence imaging agent to determine a preferred dose of the fluorescence imaging agent for imaging the anatomy or pathology of interest.

Optionally, the color image was generated by a surgical visualization system, the measure of visual enhancement is associated with a first configuration of the surgical visualization system, and the method further comprises comparing the measure of visual enhancement associated with the first configuration of the surgical visualization system with a measure of visual enhancement associated with a second configuration of the surgical visualization system to determine a preferred configuration of the surgical visualization system for image-guided surgery of the anatomy or pathology of interest.

Optionally, the method includes determining the contribution of the first color relative to other colors in the at least one first region by calculating a first ratio of a measure of signal strength of the first color to a sum of measures of signal strengths of other colors in the at least one first region.

Optionally, the method includes determining the contribution of the first color relative to the other colors in the at least one second region by calculating a second ratio of a measure of signal strength of the first color to a sum of measures of signal strengths of other colors in the at least one second region, and wherein the measure of visual enhancement comprises a ratio of the first ratio to the second ratio.

Optionally, the measure of signal strength of the first color is a mean of signal strengths of the first color in the at least one first region and the sum of measures of signal strengths of the other colors is a sum of means of signal strengths of the other colors in the at least one first region.

Optionally, the first color is green and the other colors are red and blue or the first color is cyan and the other colors are yellow and magenta.

Optionally, the method includes comparing the measure of visual enhancement of the anatomy or pathology of interest in the color image with a measure of visual enhancement of the anatomy or pathology of interest in a second color image, wherein the second image does not include a contribution from the fluorescence imaging agent because the fluorescence imaging agent was not present in the at least a portion of anatomy or pathology of interest, the fluorescence imaging agent was not excited with fluorescence excitation light, or fluorescence imaging data was not used to generate the second image.

Optionally, identifying the at least one first region of the color image and the at least one second region of the color image comprises identifying a portion of the color image that includes the portion of the anatomy or pathology of interest and comparing pixel intensities of the first color within the portion of the color image to identify the at least one first region and the at least one second region.

Optionally, identifying a portion of the color image comprises positioning a line that extends at least partially across the anatomy or pathology of interest in the color image.

Optionally, the anatomy or pathology of interest comprises a vessel, which may be a ureter. The color image may be an endoscopic image. The fluorescence imaging agent may emit light in the infrared spectrum. The fluorescence imaging agent may be pudexacianinium chloride.

According to an aspect, a method for assessing fluorescence imaging agent-based visual enhancement in a color image in which a fluorescence imaging agent signal is represented by a first color includes displaying the color image on a display to a user; receiving, by a computing system, an input e.g. from the user corresponding to selection of at least one first region of the color image that is encompassed by a portion of anatomy or pathology of interest in which the fluorescence imaging agent is present; receiving, by the computing system, an input, e.g. from the user, corresponding to selection of at least one second region of the color image that is not encompassed by the anatomy or pathology of interest; and computing, by the computing system, a measure of visual enhancement of the anatomy or pathology of interest in the color image based on a contribution of the first color relative to other colors in the at least one first region to a contribution of the first color relative to the other colors in the at least one second region.

Optionally, the method includes illuminating tissue that comprises the anatomy or pathology of interest with fluorescence excitation light generated by an illumination system for causing fluorescence emission by the fluorescence imaging agent and with visible light generated by the illumination system; detecting fluorescence emission and reflected light from the tissue by an image sensor assembly; and generating the color image based on the light detected by the image sensor assembly.

Optionally, the measure of visual enhancement is associated with a first dose of the fluorescence imaging agent, and the method further comprises comparing the measure of visual enhancement associated with the first dose with a measure of visual enhancement associated with a second dose of the fluorescence imaging agent to determine a preferred dose of the fluorescence imaging agent for imaging the anatomy or pathology of interest.

Optionally, the color image was generated by a surgical visualization system, the measure of visual enhancement is associated with a first configuration of the surgical visualization system, and the method further comprises comparing the measure of visual enhancement associated with the first configuration of the surgical visualization system with a measure of visual enhancement associated with a second configuration of the surgical visualization system to determine a preferred configuration of the surgical visualization system for image-guided surgery of the anatomy or pathology of interest.

Optionally, the method includes computing the contribution of the first color relative to other colors in the at least one first region by calculating a first ratio of a measure of signal strength of the first color to a sum of measures of signal strengths of other colors in the at least one first region.

Optionally, the method includes computing the contribution of the first color relative to the other colors in the at least one second region by calculating a second ratio of a measure of signal strength of the first color to a sum of measures of signal strengths of other colors in the at least one second region, and wherein the measure of visual enhancement comprises a ratio of the first ratio to the second ratio.

Optionally, the measure of signal strength of the first color is a mean of signal strengths of the first color in the at least one first region and the sum of measures of signal strengths of the other colors is a sum of means of signal strengths of the other colors in the at least one first region.

Optionally, the first color is green and the other colors are red and blue or the first color is cyan and the other colors are yellow and magenta.

Optionally, receiving inputs from the user corresponding to selections of the at least one first region of the color image and the at least one second region of the color image comprises receiving an input from the user corresponding to selection of a portion of the color image that includes the portion of the anatomy or pathology of interest and displaying to the user pixel intensities of the first color within the selected portion of the color image.

Optionally, the input from the user corresponding to selection of a portion of the color image comprises the user positioning a line that extends at least partially across the anatomy or pathology of interest in the displayed color image.

Optionally, the anatomy or pathology of interest comprises a vessel.

Optionally, the vessel comprises a ureter.

Optionally, the color image is an endoscopic image.

Optionally, the fluorescence imaging agent emits light in the infrared spectrum. Optionally, the fluorescence imaging agent is pudexacianinium chloride.

According to an aspect, a system for assessing fluorescence imaging agent-based visual enhancement in a color image in which a fluorescence imaging agent signal is represented by a first color includes an image guided surgery system that includes: an illuminator configured to illuminate tissue that comprises the anatomy or pathology of interest with fluorescence excitation light generated for causing fluorescence emission by the fluorescence imaging agent and with visible light, an image sensor assembly configured to detect fluorescence emission and reflected light from the tissue, and an image processing system configured to generate the color image; and a computing system comprising a display, one or more processors, memory, and one or more programs stored in the memory and including instruction for execution by the one or more processors for causing the computing system to: display the color image on the display to a user, receive an input (e.g. from the user) corresponding to selection of at least one first region of the color image that is encompassed by a portion of anatomy or pathology of interest in which the fluorescence imaging agent is present, receive an input (e.g. from the user) corresponding to selection of at least one second region of the color image that is not encompassed by the anatomy or pathology of interest, and compute a measure of visual enhancement of the anatomy or pathology of interest in the color image based on a contribution of the first color relative to other colors in the at least one first region to a contribution of the first color relative to the other colors in the at least one second region.

Optionally, the one or more programs include instructions for computing the contribution of the first color relative to other colors in the at least one first region by calculating a first ratio of a measure of signal strength of the first color to a sum of measures of signal strengths of other colors in the at least one first region.

Optionally, the one or more programs include instructions for computing the contribution of the first color relative to the other colors in the at least one second region by calculating a second ratio of a measure of signal strength of the first color to a sum of measures of signal strengths of other colors in the at least one second region, wherein the measure of visual enhancement comprises a ratio of the first ratio to the second ratio.

Optionally, the measure of signal strength of the first color is a mean of signal strengths of the first color in the at least one first region and the sum of measures of signal strengths of the other colors is a sum of means of signal strengths of the other colors in the at least one first region.

Optionally, the first color is green and the other colors are red and blue or the first color is cyan and the other colors are yellow and magenta.

Optionally, receiving inputs (e.g. from the user) corresponding to selections of the at least one first region of the color image and the at least one second region of the color image comprises receiving an input (e.g. from the user) corresponding to selection of a portion of the color image that includes the portion of the anatomy or pathology of interest and displaying to the user pixel intensities of the first color within the selected portion of the color image.

Optionally, the input from the user corresponding to selection of a portion of the color image comprises the user positioning a line that extends at least partially across the anatomy or pathology of interest in displayed the color image.

Optionally, the anatomy or pathology of interest comprises a vessel, such as a ureter. The color image may be an endoscopic image. The fluorescence imaging agent may emit light in the infrared spectrum. The fluorescence imaging agent may be pudexacianinium chloride.

According to an aspect, a method for determining a relationship between a fluorescence imaging agent-based visual enhancement in a color image of a patient and the concentration of the fluorescence imaging agent in a biological sample from the patient, wherein a fluorescence imaging agent signal in the color image is represented by a first color includes: identifying at least one first region of the color image that is encompassed by a portion of anatomy or pathology of interest in which the fluorescence imaging agent is present; identifying at least one second region of the color image that is not encompassed by the anatomy or pathology of interest; determining a measure of visual enhancement of the anatomy or pathology of interest in the color image by comparing a contribution of the first color relative to other colors in the at least one first region to a contribution of the first color relative to the other colors in the at least one second region; and calculating the relationship between the fluorescence imaging agent-based visual enhancement in the color image and the concentration of the fluorescence imaging agent in the biological sample.

Optionally, the measure of visual enhancement is determined at two or more different dosage amounts of the fluorescence imaging agent, and calculating the relationship comprises comparing the fluorescence imaging agent-based visual enhancement in the color image to the concentration of the fluorescence imaging agent in the biological sample at each of the two or more dosage amounts.

Optionally, determining the relationship comprises determining a dosage range over which there is a positive linear correlation between the fluorescence imaging agent-based visual enhancement in a color image of a patient and the concentration of the fluorescence imaging agent in a biological sample from the patient.

Optionally, the biological sample comprises urine, blood, lymphatic fluid, or feces.

According to an aspect, a system for assessing fluorescent imaging agent-based visual enhancement in a color image includes: one or more processors; and memory communicatively coupled with the one or more processors, the memory storing instructions that, when executed by the one or more processors, cause the one or more processors to: analyze pixel data of the color image to identify a first region of the color image associated with presence of a fluorescence imaging agent and a second region of the color image associated with lack of presence of the fluorescence imaging agent, the first region corresponding to a portion of anatomy or pathology of interest, and the fluorescence imaging agent being represented in a first color of the pixel data, calculate a first contribution value for the first region based on pixel values of the first region that are associated with the first color and pixel values of the first region that are associated with at least one other color and a second contribution value for the second region based on pixel values of the second region that are associated with the first color and pixel values of the second region that are associated with the at least one other color, and determine a visual enhancement value associated with the fluorescence imaging agent being present in the first region in the color image by comparing the first contribution value to the second contribution value.

Optionally, the visual enhancement value is included as part of a five-point visual enhancement scale indicating a visibility improvement of the first region of the anatomy or pathology of interest based on presence of the fluorescent imaging agent.

Optionally, the visual enhancement value is included as part of a three-point visual enhancement scale indicating a visibility improvement of the first region of the anatomy or pathology of interest based on presence of the fluorescent imaging agent.

Optionally, the instructions, when executed by the one or more processors, further cause the one or more processors to identify the first region and the second region of the color image by: comparing a pixel value for the first color of one or more first pixels in the first region to a first pixel color value threshold and a pixel value of the first color of one or more second pixels in the second region to the first pixel color value threshold, and determining that the pixel value for the first color of one or more first pixels exceeds the first pixel color value threshold and that pixel value of the first color of the one or more second pixels is less than the first pixel color value threshold.

Optionally, the instructions, when executed by the one or more processors, further cause the one or more processors to combine fluorescent pixel data of a fluorescent image of the anatomy or pathology of interest with white light pixel data of a white light image of the anatomy or pathology of interest to generate the pixel data of the color image.

Optionally, in any of the methods and systems above, the measure of visual enhancement comprises a measure of the perceived conspicuity of the anatomy or pathology of interest.

According to an aspect, a non-transitory computer readable medium.

It will be appreciated that any one or more of the above variations, aspects, features and options can be combined.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described, by way of example only, with reference to the accompanying drawings, in which:

FIG. 1A illustrates an exemplary surgical visualization system;

FIG. 1B illustrates aspects of an exemplary light source assembly;

FIG. 1C illustrates aspects of an exemplary image acquisition assembly;

FIG. 2 is a flow diagram of an exemplary method for assessing fluorescence imaging agent-based visual enhancement in a color image or video frame;

FIG. 3 is an example of an image that captures anatomy of interest at a time when a fluorescence imaging agent is present in the anatomy of interest;

FIG. 4 is an exemplary graph of pixel values versus pixel location in an initial search region of a color image or video frame;

FIG. 5 includes two color images or video frames of anatomy of interest of a subject, one capturing the anatomy of interest when a signal from the fluorescence imaging agent is present and the other capturing the anatomy of interest when a signal from the fluorescence imaging agent is absent;

FIG. 6 shows the bitmaps of the red, green, and blue color components for each of the color images or video frames of FIG. 5;

FIGS. 7A-7C are graphs that plot the signal intensity values for each color component for a set of identified pixels in the color images of FIG. 5;

FIG. 7D is a graph of the signal intensity values of the green component illustrating regions that are located within and outside of the anatomy of interest;

FIG. 8 is a flow diagram of an exemplary method for using a measure of contrast enhancement;

FIG. 9 is a functional block diagram of an exemplary computing system;

FIG. 10 shows box plots of a contrast enhancement factor throughout time points according to an example;

FIG. 11 shows box plots of a correlation between a surgeon's qualitative assessment of fluorescence intensity and a quantitative assessment generated according to an example; and

FIG. 12 shows contrast enhancement factor distribution data by dose, according to an example.

### DETAILED DESCRIPTION

Reference will now be made in detail to implementations and examples of various aspects and variations of methods described herein. Although several exemplary variations of the methods are described herein, other variations of the methods may include aspects of the methods described herein combined in any suitable manner having combinations of all or some of the aspects described.

Disclosed herein are methods for quantifying the color contrast provided by fluorescence imaging agents utilized before, during, and/or after image-guided surgery. Quantifying such color contrast can be important for (but is not limited to) quantifying a user's ability to visualize an anatomical feature, for determining the near optimal or optimal dose and/or administration timing of the fluorescence imaging agent, as a biomarker of a surgeon's ability to visualize an anatomical feature, for determining the relative performance of various fluorescence imaging agents in highlighting tissue areas of interest, for determining optimal operational parameters of video technology, and/or for determining an appropriate visualization protocol during image-guided surgery.

A color image (the terms image, video frame, and video image are used interchangeably throughout) capturing anatomy or pathology of interest at a time when a fluorescence imaging agent is present in the anatomy or pathology of interest may be analyzed to quantify the contrast between the anatomy or pathology of interest containing the fluorescence imaging agent and the surrounding tissue. The fluorescence imaging agent may have been pre-administered. Such color images are typically composed of both reflected light signals and fluorescence light signals, where the fluorescence light signal from the fluorescence imaging agent is represented by a particular color. The color used to represent the fluorescence imaging agent signal is typically chosen to align with a color component of a color image.

Color components may refer to the primary color components (such as red, green, and blue components) into which a color image may be decomposed. Alternately, color components may refer the complementary color components (cyan, magenta, yellow) or some other color model for which components could also be combined to form a color image.

For a set of image pixels in such a color image corresponding to the anatomy or pathology of interest containing the fluorescence imaging agent, the normalized, average signal strength of the color component used to represent the fluorescence imaging agent in the color image can be calculated by dividing the average signal strength for that color component by the sum of the average signal strengths of one or more other color components for those same pixels. A similar normalized, average signal strength can be calculated for image pixels corresponding to the tissue surrounding the anatomy or pathology of interest in which no fluorescence imaging agent is present. A color contrast between the anatomy or pathology of interest containing the fluorescence imaging agent and the surrounding tissue can then be calculated as a ratio of these two normalized average signal strengths. Such a quantification of color contrast can similarly be computed and compared for images of the anatomy or pathology of interest when evaluating different fluorescence imaging agent doses, when comparing the performance of different fluorescence imaging agents, and/or when different visualization systems or system settings are being compared.

A similar analysis and computation to quantify the color contrast between the anatomy or pathology of interest and the surrounding tissue may be applied to a color video image when no signal from the fluorescence imaging agent is present in the anatomy or pathology of interest. If the primary source of color contrast is due to the signal from the fluorescence imaging agent, a color contrast analysis and computation between the anatomy or pathology of interest and the surrounding tissue as described above will typically return a value substantially close to unity when no signal from the fluorescence imaging agent is present in the anatomy or pathology of interest.

Although the calculated color contrast between the anatomy or pathology of interest and the surrounding tissue may be substantially close to unity when no signal from the fluorescence imaging agent is present, the calculated color contrast is not always exactly unity. In this case, a contrast enhancement factor can be determined by computing a ratio of the quantified color contrast between the anatomy or pathology of interest and the surrounding tissue when a signal from the fluorescence imaging agent is present to the color contrast between the anatomy or pathology of interest and the surrounding tissue when no signal from the fluorescence imaging agent is present. The contrast enhancement factor provides a measure of the degree to which the presence of a signal from the fluorescence imaging agent in the anatomy or pathology of interest contributes to the color contrast perceived by the surgeon while normalizing the contributions to the color contrast value by other factors. In clinical terms, the contrast enhancement factor can be a measure of the perceived conspicuity of the target anatomy or pathology, which may be characterized by the proportion of fluorescent light signal to reflected light signal or may alternately be characterized as the identification of the target anatomy or pathology location.

For example, a contrast enhancement factor may be computed for a pair of images that capture substantially similar scenes at times when the fluorescence imaging agent is present in the anatomy or pathology of interest and when the fluorescence imaging agent is not present in the anatomy or pathology of interest, and the color contrast quantification can be performed for both images. The contrast enhancement factor computed from the pair of images can be compared to the contrast enhancement factor computed from one or more other pairs of images corresponding to, different fluorescence imaging agent doses, different fluorescence imaging agents, and/or different visualization systems or system settings to assess which fluorescence imaging agent dose, fluorescence imaging agent type, visualization system, and/or visualization system settings are optimal for visualizing the anatomy or pathology of interest.

Alternatively, color contrast between the anatomy or pathology of interest and surrounding tissues and a contrast enhancement factor may be computed for a pair images that capture the same scene or substantially similar scenes when the fluorescence imaging agent is present in the anatomy or pathology of interest, but in which a first color image includes a signal from the fluorescence imaging agent and a second color image does not include a signal from the fluorescence imaging agent. This can be done, for example, by generating the second color image without using the signal from the fluorescence imaging agent (e.g., the first color image is generated from reflected light signals and a fluorescence light signal and the second color image is generated from just the reflected light signals) or by generating the second color image from imaging data captured during a period in which the anatomy or pathology of interest is not illuminated with fluorescence excitation illumination. In these cases, the presence or absence of signal from the fluorescence imaging agent is not due to the presence or absence of the fluorescence imaging agent in the anatomy or pathology of interest but due to the fluorescence imaging agent not being excited or the signal from the fluorescence imaging agent not being used.

The color contrast between the anatomy or pathology of interest and surrounding tissues may be computed for a color image that captures a scene of a patient, e.g. before, during, or after surgery, wherein the color image does not include a signal from fluorescence (e.g. because no fluorescence imaging agent is present in the anatomy or pathology of interest). Based on a desired contrast enhancement factor, a determination can be made which fluorescence imaging agent dose, fluorescence imaging agent type, visualization system, and/or visualization system settings are optimal for visualizing the anatomy or pathology of interest in the scene of the patient. Any method can include selecting the optimal fluorescence imaging agent dose, fluorescence imaging agent type, visualization system, and/or visualization system settings on the basis of the desired contrast enhancement factor. Any method can include applying the selected optimal fluorescence imaging agent dose, fluorescence imaging agent type, visualization system, and/or visualization system settings while capturing an image of the scene of the patient including a signal from fluorescence, e.g. at times when the fluorescence imaging agent is present, e.g. pre-administered, in the anatomy or pathology of interest and/or the anatomy or pathology of interest is illuminated with fluorescence excitation illumination.

In the following description, it is to be understood that the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

### Surgical Visualization System

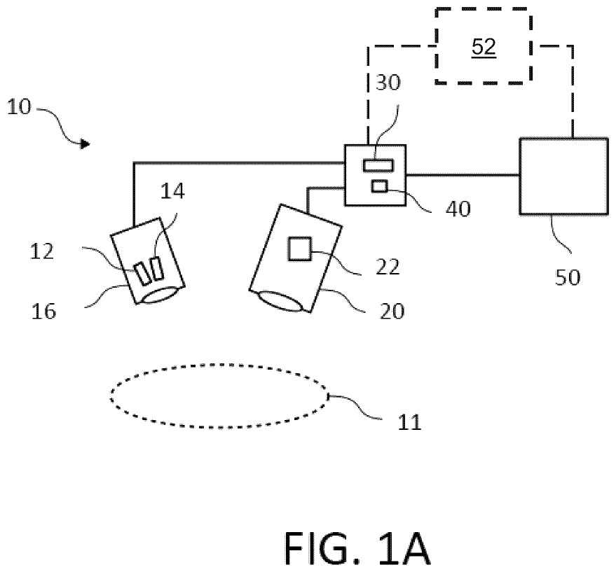

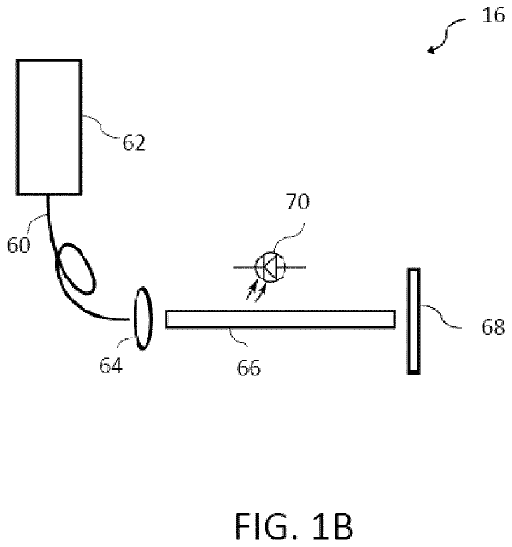

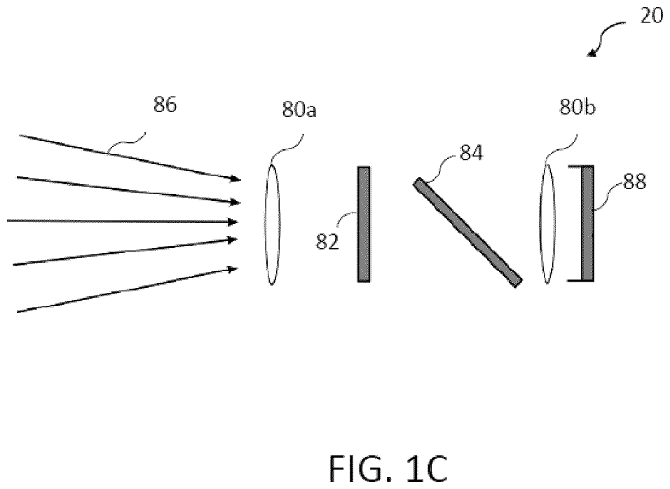

FIG. 1A illustrates an exemplary surgical visualization system 10 for imaging tissue 11 (e.g., a region of interest) in at least two different spectral bands and generating color images composed of both reflected light signals and fluorescence light signals, where the fluorescence light signal from the fluorescence imaging agent is represented by a particular color. System 10 may include an image acquisition assembly 20 that includes at least one image sensor 22 configured to acquire a sequence of video frames depicting the tissue and/or one or more features of the tissue.

As shown in the schematic of FIG. 1A, the system 10 may include a light source assembly 16 (also referred to herein as an illuminator) including at least two light sources that emit light of different spectral bands. The light sources can be, for example, a light source 12 that emits light for illumination during reflected light imaging (e.g., full spectrum visible light, narrow band light, or other portions of the light spectrum not intended to excite fluorescence) and an excitation light source 14 that emits excitation light for illuminating and exciting fluorophores in the tissue 11 and causing fluorescence emission. In some examples, the fluorescence excitation light can be in a waveband outside the visible light spectrum, while in other examples the fluorescence excitation light can be in a waveband within or overlapping the visible light spectrum.

The visible light source 42 is configured to emit visible light for illumination of the object to be imaged. In some variations, the visible light source may include one or more solid state emitters, such as LEDs and/or laser diodes. For example, the visible light source may include blue, green, and red (or other color components) LEDs or laser diodes that in combination generate white light illumination. These color component light sources may be centered around the same wavelengths around which the image acquisition assembly (described further below) is centered. For example, in variations in which the image acquisition assembly includes a single chip, single color image sensor having an RGB color filter array deposited on its pixels, the red, green, and blue light sources may be centered around the same wavelengths around which the RGB color filter array is centered. As another example, in variations in which the image acquisition assembly includes a three-chip, three-sensor (RGB) color camera system, the red, green, and blue light sources may be centered around the same wavelengths around which the red, green, and blue image sensors are centered.

The excitation light source 14 is configured to emit excitation light suitable for exciting intrinsic fluorophores and/or extrinsic fluorophores (e.g., a fluorescence imaging agent introduced into the object) located in the tissue being imaged. The excitation light source 14 may include, for example, one or more LEDs, laser diodes, arc lamps, and/or illuminating technologies of sufficient intensity and appropriate wavelength to excite the fluorophores located in the object being imaged. For example, the excitation light source may be configured to emit light in the near-infrared (NIR) waveband (such as, for example, approximately 805 nm light), though other excitation light wavelengths may be appropriate depending on the application. As another example, the excitation light source may be configured to emit light in a lower waveband such as to excite a fluorophore with an emission waveband within or overlapping the visible spectrum.

The light source assembly 16 can be configured for generating any desired spectral bands, including more than two spectral bands. For example, the light source assembly 16 can be configured to provide two different excitation wavebands for exciting two or more different types of fluorophores in the tissue (with or without also providing visible light).

The light source assembly 16 may further include one or more optical elements that shape and/or guide the light output from the visible light source 12 and/or excitation light source 14. The optical components may include one or more lenses, mirrors (e.g., dichroic mirrors), light guides and/or diffractive elements, e.g., so as to help ensure a flat field over substantially the entire field of view of the image acquisition assembly 20. For example, as shown in the schematic of FIG. 1B, the output 60 from a laser diode 62 (providing visible light or excitation light) may be passed through one or more focusing lenses 64, and then through a light guide 66. The light may be further passed through an optical diffractive element 68 (e.g., one or more optical diffusers). Power to the laser diode 62 may be provided by, for example, a high-current laser driver and may optionally be operated in a pulsed mode during the image acquisition process according to a timing scheme. An optical sensor such as a solid state photodiode 70 may be incorporated into the light source assembly and may sample the illumination intensity produced by one or more of the light sources, via scattered or diffuse reflections from the various optical elements.

The image acquisition assembly 20 may acquire reflected light video frames based on visible light that has reflected from the object, and/or fluorescence video frames based on fluorescence emitted by fluorophores in the object that are excited by the fluorescence excitation light. As shown in FIG. 1C, the image acquisition assembly 20 may acquire images using a system of optics (e.g., one or more lenses 80a and 80b, one or more filters 82, and one or more mirrors 84, beam splitters, etc.) to collect and focus reflected light and/or fluorescence light 86 onto an image sensor assembly 88. The image sensor assembly 644 may include at least one solid state image sensor. The one or more image sensors may include, for example, a charge coupled device (CCD), a CMOS sensor, a CID, or other suitable sensor technology. The image sensor assembly 88 may include a single chip, single image sensor (e.g., a grayscale image sensor or a color image sensor having an RGB color filter array deposited on its pixels) with appropriate sensitivity to acquire image signals across the visible and NIR bands spanning the reflected illumination light and fluorescence emission light spectrum. In another variation, the image acquisition assembly may include a three-chip, three-sensor (RGB) image sensor assembly 88 or other multi-sensor assembly (e.g., a 4-chip RGBNIR sensor assembly or a 2-chip color and NIR sensor assembly).

As shown in the schematic of FIG. 1A, the system 10 may include one or more image processors 30. The one or more image processors 30 may include, for example, any suitable microprocessor or combination of microprocessors, including one or more central processing unit and/or one or more graphics processing units. The one or more image processors 30 may be configured to execute instructions for generating color video images from reflected light video frames and fluorescence video frames acquired by the image acquisition assembly 20 in which the fluorescence signal is represented by one of the colors used for the color video image. The color video images may include red, green, and blue pixel intensity values, cyan, magenta, and yellow pixel intensity values, or pixel intensity values according to any other color model. As visible light frames, fluorescence frames, and/or color images generated from visible light and fluorescence frames are acquired and/or generated, at least a portion may be displayed such as for image-guided surgery and/or may be stored by the one or more image processors 30 in memory for retrieval for the analyses described herein.

As shown in the schematic of FIG. 1A, the system 10 may include a controller 40, which may be embodied in, for example, a microprocessor and/or timing electronics. In some variations, a single image sensor may be used to acquire both visible light video frames and fluorescence frames, and the controller 40 may control a timing scheme for the visible light source 12 and/or the excitation light source 14, and the image acquisition assembly 20. This timing scheme may enable separation of the image signal associated with the visible light signal and the image signal associated with the fluorescence signal. In particular, the timing scheme may involve illuminating the object with light for reflected light imaging and/or excitation light for fluorescence emission imaging according to a pulsing scheme and processing the visible light image signal and fluorescence image signal with a processing scheme, wherein the processing scheme is synchronized and matched to the pulsing scheme (e.g., via a controller) to enable separation of the two image signals in a time-division multiplexed manner. Examples of such pulsing and image processing schemes have been described in U.S. Pat. No. 9,173,554, filed on Mar. 18, 2009 and titled “IMAGING SYSTEM FOR COMBINED FULL-COLOR REFLECTANCE AND NEAR-INFRARED IMAGING,” the contents of which are incorporated in their entirety by this reference. However, other suitable pulsing and image processing schemes may be used to acquire reference video frames and low light video frames simultaneously, for example to acquire reflected light video frames and fluorescence video frames simultaneously. Furthermore, the controller may be configured to control the timing scheme for the visible light source and/or the excitation light source, and the image acquisition assembly based at least in part on the relative movement between the image acquisition assembly and the object.

In general, the processing scheme in the image processor 30 is synchronized and matched to the pulsing scheme in the light source assembly 16 (e.g., via the controller 40) to enable the separation of the image signal associated with reflected light from the image signal associated with the fluorescence emission. The rate of pulsing and image processing may be such that the processed image signals are output for display and/or saving in real time (i.e., with negligible latency).

The image data from the reflected light may be processed by any suitable color image processing methods. For example, in variations having an image sensor with a color filter array deposited on the sensor surface, image processing may include de-mosaicing the color image signal, followed by amplification, A/D conversion, and/or storage in color image memory. The typical (but not the only) signal format after such processing is luminance/chrominance (Y_{c}, cᵣc_{b}) format. In variations having three solid state image sensors mounted on a Philips (RGB) prism (or other beam splitting element), image processing may include receiving a direct readout of the red, green, and blue color image from the camera, followed by amplification, A/D conversion, and/or storage in color image memory. The typical (but not the only) signal format after such processing is luminance/chrominance (Y_{c}, cᵣc_{b}) format.

The processed image data may be output to a display and/or recorded in high definition (HD) or ultra-high definition (UHD or 4K) resolution (or any suitable resolution), with negligible latency. The color image data and fluorescence image data may be simultaneously output in separate channels for display and/or recording. Displayed and/or recorded reflected light image data may have a high color fidelity, such that it is a highly accurate color depiction of the surface that is reflecting the light. Reflected light image data and fluorescence image data may be overlaid or otherwise combined. For example, the fluorescence emission image data may be used to modify the chrominance (cᵣ, c_{b})in the white light image data such that pixels with higher fluorescence signal intensity are increasingly saturated by a non-naturally occurring color (e.g., green in biological systems).

The system 10 may include one or more data modules 50 that communicates and/or stores some or all of the acquired frames and/or information generated from the image data. For instance, the data module 50 may include a display (e.g., computer screen or other monitor), recorder or other data storage device, and/or picture archiving and communication system (PACS). The system may additionally or alternatively include any suitable systems for communicating and/or storing images and image-related data. The system 10 may be communicatively connected (e.g., via one or more local and/or remote networks) to one or more external computing systems 52, which may receive one or more images generated by system 10 for further processing and analysis according to the principles described herein. The one or more external computing systems 52 may receive one or more images from storage of the system 10 (e.g., data module 50) or directly from the one or more image processors 30.

In general, the system 10 may be used in conjunction with a range of surgical and non-surgical methods. For example, the imaging system 10 may be used during non-invasive imaging sessions that may or may not include treatment, in “open” surgical procedures, or during minimally invasive surgical procedures, sometimes referred to as band aid or keyhole surgeries. In open procedures, an incision sufficiently large to expose the entire operative area is made with a scalpel or other knife and tissue of interest may be imaged using an imaging system 10 configured as an open-field imaging system. In minimally invasive surgeries, one or more much smaller incisions are typically made, through which a laparoscope and/or other endoscopic tools of an endoscopic imaging system 10 may be inserted to allow a surgeon to view and/or surgically manipulate a patient's organs and/or tissues.

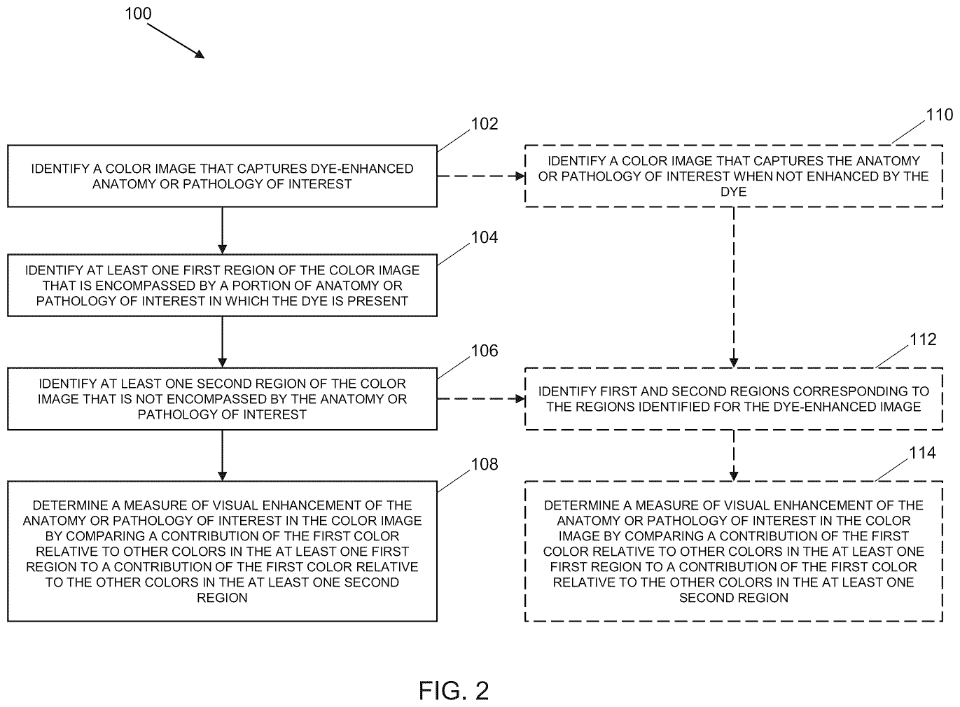

FIG. 2 is a flow diagram of a method 100 for assessing fluorescence imaging agent-based visual enhancement in a color image or video frame (the term color image is used in the following for simplicity but should be understood as encompassing a video frame) generated by a surgical visualization system, such as system 10 of FIG. 1A. Method 100 can be used to quantify the contrast provided by a fluorescence imaging agent in the color image. Contrast quantifications (also referred to herein as contrast enhancement factors) for different images can be compared to assess the relative degree of visual enhancement provided by a particular fluorescence imaging agent, fluorescence imaging agent dose, visualization system and/or visualization system settings. Method 100 can be performed for any color image or color video frame.

At step 102, a color image of a subject is identified that captures within it the anatomy or pathology of interest in which a visually enhancing fluorescence imaging agent is present. The color image may have been generated prior to start of the method 100. The anatomy or pathology of interest can be any type that can be visually enhanced via a fluorescence imaging agent. For example, the anatomy or pathology of interest can be perfused tissue, blood vessels, lymph nodes, ureters, bile ducts, nerves, tumors, lesions, diseased tissues, etc. For example, the color image identified at step 102 may capture one or more vessels (e.g., a blood vessel, a ureter) at a time when a fluorescence imaging agent is present in the vessel. The fluorescence imaging agent may have been administered prior to start of the method 100. The color image can be a single exposure snapshot, or a frame extracted from continuous video. The color image may represent the fluorescence imaging agent in a specific color of the color image. For example, the color image may include red, green, and blue color components, and the fluorescence imaging agent may be represented as a green signal contributing to the green component of the color image, or the color image may include cyan, magenta, yellow, and the fluorescence imaging agent may be represented as a cyan signal contributing to the cyan component of the color image.

The color image may have been generated by a surgical visualization system (e.g., system 10 of FIG. 1A) that generated the color image during a medical procedure, such as for surgical video displayed to a surgeon during image-guided surgery. The fluorescence imaging agent present in the anatomy or pathology of interest when the color image was generated may be, for example, a fluorescence imaging agent that fluoresces when excited by excitation light. A surgical visualization system may have generated the color image by capturing a white light image of the anatomy of a subject and capturing a fluorescence image of the anatomy of the subject and combining the white light image data and fluorescence image data to produce the color image such that the fluorescence image data is represented within a color component of the color image.

The color image or video frame may be identified from among a series of such images or extracted as a video frame from continuous video based on the presence or degree of presence of the fluorescence imaging agent within the anatomy or pathology of interest. For example, a user may review a video generated during a medical procedure that is displayed on a display and identify and select via a user input device of a computing system one or more frames of the video that show the fluorescence imaging agent in the anatomy or pathology of interest.

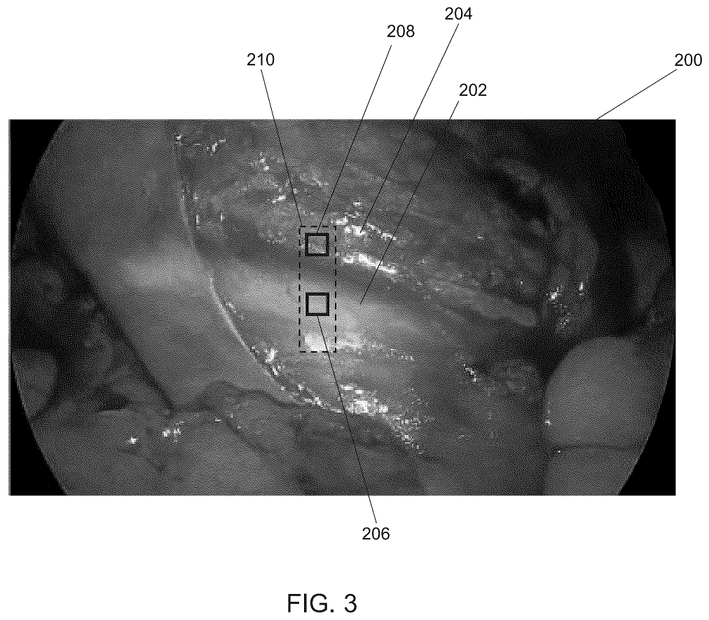

FIG. 3 is an example of a color image selected according to step 102. The exemplary color image 200 is a frame extracted from a surgical video showing tissue in the abdominal cavity of a patient. In this case, the color image 200 captures anatomy of interest (which in the example image is a ureter 202) at a time when a fluorescence imaging agent was present in the anatomy of interest. The signal from the fluorescence imaging agent is represented in green and contributes to the green component of the color image 200. Thus, bright green regions of the color image 200 corresponds to the ureter 202. The surrounding tissue 204 does not have fluorescence imaging agent in it at the time the video frame as represented by the color image 200 was acquired. As the green signal from the surrounding tissues is relatively small, the green display color for the fluorescence imaging agent fluorescence contrasts with the colors of the surrounding tissues 204 in the abdominal cavity and enables the surgeon to identify the ureter.

At step 104, at least one region of the color image is identified that is encompassed by a portion of anatomy or pathology of interest in which a visually enhancing fluorescence imaging agent is present. For example, with reference to color image 200 of FIG. 3, a region 206 is identified that is encompassed by the ureter 202. Generally, the region identifies a collection of pixels that will be used to quantify the contrast provided by the fluorescence imaging agent. In the illustrated example, the region 206 is defined by a rectangular shape, but the region can be defined in any suitable fashion, including by a circular shape, a line or line segment, a single pixel, etc. The region may be identified manually by a user, such as by viewing the color image on a display screen and defining the boundaries of the region via a computer mouse, keyboard, touch screen, etc., based on where the visually enhancing fluorescence imaging agent is located in the color image. Optionally, the region may include a portion of the color image that has the strongest signal from the fluorescence imaging agent (e.g., the brightest region associated with the fluorescence imaging agent). Optionally, the region may be automatically identified at step 104 by a computing system that analyzes pixel data of the color image to identify a region within the color image within which a fluorescence imaging agent is present, which corresponds to a portion of the anatomy or pathology of interest. For example, the computing system may identify a region having pixel values above a threshold for a color component used to represent fluorescence in the color image and/or may implement a machine learning model trained to identify regions of strong fluorescence intensity.

The region identified in step 104 may be identified via a combination of a visual inspection of the color image to select an initial region that includes a strong fluorescence imaging agent signal with an analysis of pixel values in that region to identify the region encompassed by the anatomy of interest. For example, based on a display of the color image, an initial search region may be defined that encompasses a portion of the color image that has a strong fluorescence imaging agent signal as well as surrounding portions, and the pixel values for the color component used to show the fluorescence imaging agent signal may be analyzed to locate where the fluorescence imaging agent signal is strongest.

At step 106, at least one region of the color image that is not encompassed by the anatomy or pathology of interest is identified. For example, with reference to image 200 of FIG. 3, region 208 is identified that is not encompassed by the ureter 202 based on there being little to no fluorescence imaging agent-related signal. The region may be identified manually by a user, such as by viewing the color image on a display screen and defining the boundaries of the region via a computer mouse, keyboard, touch screen, etc., based on where the visually enhancing fluorescence imaging agent is not located in the color image. Optionally, the region may be automatically identified at step 106 by a computing system that analyzes pixel data of the color image to identify a region within the color image in which the fluorescence imaging agent is not present. For example, the computing system may identify a region (e.g., nearby the region identified in step 104) having pixel values below a threshold (the same threshold used in step 104 or a different threshold) for a color component used to represent fluorescence in the color image and/or may implement a machine learning model trained to identify regions of little or no fluorescence intensity. The region identified in step 106 can be the same size and/or shape as that identified in step 104 or can be a different size and/or shape. The region identified in step 106 can be identified in similar fashion as the region identified in step 104.

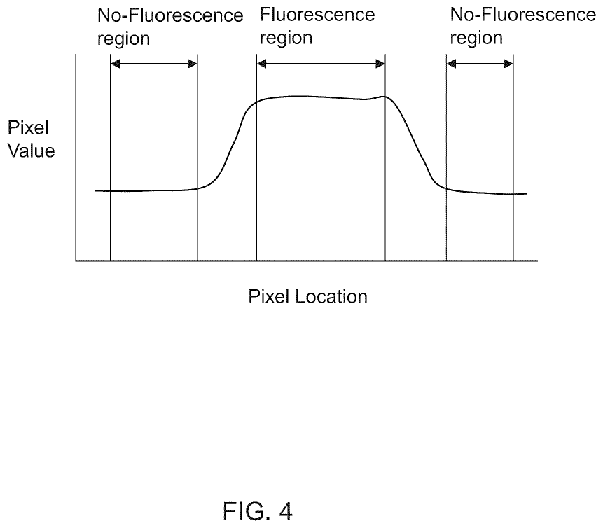

Optionally, steps 104 and 106 can include identifying the region encompassed by the anatomy or pathology of interest and the region not encompassed by the anatomy or pathology of interest at least in part by analyzing the pixel intensity values associated with the color used for representing the fluorescence imaging agent in the color image to locate one or more regions of pixels that are strongly correlated with the presence of fluorescence imaging agent. For example, an initial search region 210 may be selected in the color image 200 based on visual inspection of the color image 200, such as via one or more user inputs to a computing system that select the initial search region 210. The initial search region 210 may be selected to encompass a continuous portion of the color image that includes a region with a strong fluorescence imaging agent signal as well as surrounding regions. The pixel values in the initial search region 210 for the color component used for representing the fluorescence imaging agent (e.g., green) may then be inspected to identify at least one region encompassed by the anatomy of interest and at least one region not encompassed by the anatomy of interest. For example, a graph of pixel values versus pixel location in the initial search region 210 for the color component associated with the fluorescence imaging agent (e.g., green) may be plotted, as shown in FIG. 4, and displayed to the user, and the pixel locations associated with the highest pixel values—indicating the presence of fluorescence imaging agent—may be selected (e.g., by the user via a user input to a computing system or automatically by the computing system) for step 104 as the region that is encompassed by the anatomy of interest. Pixel locations that have more moderate pixel values relative to that of the region encompassed by the anatomy of interest—indicating the absence of fluorescence imaging agent—may be selected (e.g., by the user via a user input to a computing system or automatically by the computing system) for step 106 as the region not encompassed by the anatomy of interest. As illustrated in the example of FIG. 4, more than one region can be selected for step 106—a “no-fluorescence” region on either side of the fluorescence region is identified.

Returning to FIG. 1A, method 100 continues with step 108 in which a measure of visual enhancement (also referred to herein as a visual enhancement value) of the anatomy of interest in the color image is determined by comparing (a) a relative contribution of the color used to represent the fluorescence imaging agent in the region encompassed by the anatomy of interest to (b) a relative contribution of the color used to represent the fluorescence imaging agent in the region not encompassed by the anatomy of interest. The relative contribution of the color used to represent the fluorescence imaging agent can be the signal strength of that color relative to the signal strengths of the other colors in the region. For example, with reference to color image 200 of FIG. 3 in which the fluorescence imaging agent signal is represented in the green component of the color image, the contribution of the green signal relative to the red and blue signals in region 206 is compared to the contribution of the green signal relative to the red and blue signals in region 208 to compute a measure of the visual enhancement provided by the fluorescence imaging agent in the color image 200.

Step 108 may include generating a measure of signal intensity for each of the color components of the color image for each of the two regions, which may then be used to compute the relative contribution of the fluorescence imaging agent-containing color component to the one or more of the other color components. The measure of signal intensity for each color can be, for example, the mean intensity value for the pixels of the region for each color component, determined according to the following:

Mean signal value=(Σgrey scale values)÷n positions

Thus, the means signal value for a given color component for a respective region (or group of regions) can be the sum of the pixel intensity values divided by the number of pixels. A mean signal value could be calculated for each of the color components for the region or regions that are encompassed by the anatomy of interest (e.g., region 206 of image 200) and a mean signal value could be calculated for each of the color components for the region or regions that are not encompassed by the anatomy of interest (e.g., region 208 of image 200).

Step 108 may also include computing the relative contribution of the fluorescence imaging agent-containing color component (e.g., the green color component for the example image 200 of FIG. 3) to at least one of the other color components using the mean signal values computed as described above. The relative contribution can be computed by normalizing the mean signal value for the fluorescence imaging agent-containing color component to the sum of the mean signal values for one or more of the other color components, as follows:

$$\\ Relative Contribution of Color 1 = \frac{{C}_{1}}{{C}_{2}} or$$

$$\\ Relative Contribution of Color 1 = \frac{{C}_{1}}{{C}_{2} + {C}_{3}}$$

where, C₁ is the mean signal value for the color component used to represent the fluorescence imaging agent signal and C₂ and C₃ are the mean signal values for the other color components. For example, for the example of FIG. 3, the mean signal value for the green component for region 206 can be divided by the sum of the mean signal values for the blue component and the red component for region 206 to compute a relative contribution value for region 206. The same relative contribution calculation can be performed for region 208 to compute a relative contribution value for region 208.

As some color contrast between the anatomy of interest and the surrounding tissue may exist naturally, the degree to which the contrast is enhanced by the presence of the fluorescence imaging agent in the anatomy of interest may be quantified by additionally calculating a ratio of the relative contribution of the fluorescence imaging agent-containing color component in the region encompassed by the anatomy of interest to the relative contribution of the fluorescence imaging agent-containing color component in the region(s) not encompassed by the anatomy of interest, as follows:

$$\\ Contrast Quantification = \frac{{( Relative Contribution of Color 1 )}_{Inside}}{{( Relative Contribution of Color 1 )}_{Outside}} = \frac{{( \frac{{C}_{1}}{{C}_{2}} )}_{Inside}}{{( \frac{{C}_{1}}{{C}_{2}} )}_{Outside}}$$

$$\\ or$$

$$\\ Contrast Quantification = \frac{{( Relative Contribution of Color 1 )}_{Inside}}{{( Relative Contribution of Color 1 )}_{Outside}} = \frac{{( \frac{{C}_{1}}{{C}_{2} + {C}_{3}} )}_{Inside}}{{( \frac{{C}_{1}}{{C}_{2} + {C}_{3}} )}_{Outside}}$$

Where inside designates the region(s) encompassed by the anatomy of interest and outside designates the region(s) not encompassed by the anatomy of interest. With reference to the example image 200 of FIG. 3, the contrast quantification can be computed as follows:

$$\\ Contrast Quantification = \frac{{( \frac{G}{R + B} )}_{206}}{{( \frac{G}{R + B} )}_{208}}$$

where, R, G, and B are the mean signal values for the red, green, and blue color components, respectively, and 206 indicates the mean signal values for region 206 (the region encompassed by the ureter 202) and 208 indicates the mean signal values for region 208 (the region not encompassed by the ureter 202).

The contrast quantification provides a measure of the visual enhancement of the anatomy or pathology of interest in the color image provided by the fluorescence imaging agent, fluorescence imaging agent dose, and visualization system and system settings used for generating the color image. This measure can be generated for different fluorescence imaging agents, different fluorescence imaging agent doses, different visualization systems, and/or different visualization system settings to provide a relative assessment of those factors in the visualization of the anatomy of interest and can assist in selecting factors to achieve better visualizations of the anatomy of interest.

In at least some instances, it may be desirable to compare the measure of visual enhancement determined in step 108 with a baseline measure generated based on an image of the anatomy of interest that does not include contribution of a signal from a fluorescence imaging agent. For example, the assessment of a particular fluorescence imaging agent may include a comparison of the measure of visual enhancement for an image in which the fluorescence imaging agent is present in the anatomy of interest (a “fluorescence-containing image”) with the measure of visual enhancement for an image that does not include contribution from the fluorescence imaging agent (a “baseline” image), which provides baseline for the fluorescence imaging agent measure. The relative difference in fluorescence-containing image and baseline image measures could be compared for different fluorescence imaging agents, fluorescence imaging agent doses, visualization systems, and/or visualization system settings to assess the relative effectiveness of those factors to visualization of the anatomy of interest. In some examples, the baseline image is an image generated when there is no fluorescence imaging agent present in the anatomy of interest (e.g., an image generated before a bolus of fluorescence imaging agent has reached the anatomy of interest or a portion of the anatomy of interest that is in the field of view or after the bolus of fluorescence imaging agent has exited the anatomy of interest or a portion of the anatomy of interest in the field of view). In some examples, the baseline image is an image captured when the fluorescence imaging agent is present in the anatomy of interest but no fluorescence excitation illumination is being provided to cause the fluorescence imaging agent to fluoresce. In some examples, the baseline image is an image that is generated only from light reflected from the anatomy of interest (e.g., fluorescence from the fluorescence imaging agent is captured in a fluorescence frame but the fluorescence frame is not used for generating the baseline image). In the latter case, the fluorescence-containing image can be generated from the baseline image and a fluorescence image.

As such, method 100 may include additional steps for generating a measure of visual enhancement for a baseline image. At step 110, a “baseline” image may be selected that captures the anatomy of interest but does not include a contribution from a fluorescence imaging agent. The baseline image can be selected from the same video as the color image used in steps 102-108. The baseline image can be selected based on a similarity between the baseline image and the color image used in steps 102-108. For example, the baseline image can include the same portion of the anatomy of interest and/or include the same or similar field of view.

At step 112, regions that correspond to the regions identified in steps 104 and 106 are identified in the baseline image. This can be done manually, such as by visually comparing the baseline image to the fluorescence-containing image and locating the regions in similar regions of the anatomy as the identified regions in the fluorescence-containing image. Optionally, the fluorescence-containing image and/or baseline image can be automatically or manually adjusted (e.g., scaled, cropped, rotated, and/or shifted) so that the field of view of the color images align. Then, the same locations for the regions identified in steps 104 and 106 can be identified in the baseline image based, for example, on pixel location.

At step 114, the measure of visual enhancement is determined for the baseline image in the same fashion as it is determined for the fluorescence-containing image in step 108. The measure of visual enhancement for the baseline image can be used as a baseline for the measure of visual enhancement for the fluorescence-containing image. In instances in which the anatomy of interest is similar in visual appearance with surrounding tissue, the baseline measure of visual enhancement generated at step 114 is likely to be near 1, indicating that the relative contribution of the component used for displaying the fluorescence signal (e.g., green) is similar for both the anatomy of interest and the surrounding tissue. However, in instances in which the anatomy of interest has a different appearance from surrounding tissue, the baseline measure of visual enhancement generated at step 114 may not be near 1. As such, the measure of visual enhancement determined at step 114 can provide a baseline for the measure of visual enhancement determined at step 108. The relative differences between the contrast quantification determined at step 108 and the baseline contrast quantification determined at step 114 can be compared across different pairs of fluorescence imaging agent/baseline images for different combinations of fluorescence imaging agent, fluorescence imaging agent dose, visualization system, and/or visualization system settings to determine whether particular choices for one or more of these factors is better than other choices—e.g., whether one fluorescence imaging agent is better than another or whether one fluorescence imaging agent dose is better than another.

A quantitative assessment of the contrast enhancement provided by a fluorescence imaging agent in a color image, performed according to method 100, can provide several benefits for image-guided surgical applications. The quantitative assessment can correlate to surgeons' qualitative assessments of fluorescence intensity (surgeon's ability to identify/visualize the anatomy of interest). Thus, the quantitative assessment can serve as a substitute for (or corroboration of) surgeon qualitative assessment in evaluating fluorescence imaging agents, fluorescence imaging agent dose, and/or image-guided surgical systems or system settings. Additionally, the quantitative assessment can be used for assessing near-optimal or optimal doses of fluorescence imaging agents for use in image-guided surgery. As such, quantitative assessments according to the principles described herein provide a measure that can be used as a biomarker of surgeons' abilities to visualize and/or identify an anatomical structure as it is both translatable and unbiased.

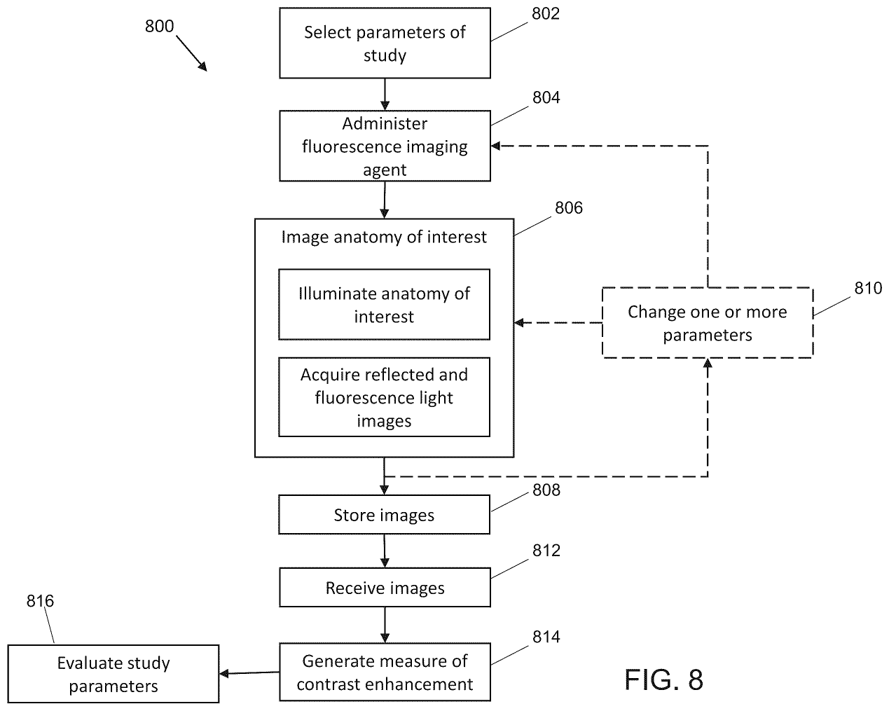

FIG. 8 illustrates a method 800 for using measures of contrast enhancement generated according to method 100. Method 800 can be performed to quantify a surgeon's observed visualization of an anatomical feature, determine an optimal or near optimal dose of a fluorescence imaging agent, generate a biomarker of a surgeon's ability to visualize and/or identify an anatomical structure, compare performances of different fluorescence imaging agents, and compare performances of different surgical visualization systems (e.g., different variations of surgical visualization system 10 of FIG. 1A or other surgical visualization systems) or different settings of a surgical visualization system (e.g., different settings of surgical visualization system 10 of FIG. 1A).

At step 802, values for one or more parameters of a fluorescence visualization study are selected according to the study goals. The parameters can include one or more of which fluorescence imaging agent to use, which dose of the fluorescence imaging agent to use, which image-guided surgical system to use, and which settings for a given image-guided surgical system to use. At step 804, a dose of a selected fluorescence imaging agent is obtained to be administered to a subject. Method 800 can exclude the step of administering the fluorescence imaging agent. The fluorescence imaging agent can be administered intravenously or through any other suitable fashion.

At step 806, the selected surgical visualization system is used to image anatomy of interest of the subject. Step 806 can include illuminating the anatomy of interest with white light and/or with fluorescence excitation light for causing the fluorescence imaging agent to fluoresce, such as via light source assembly 16 of FIG. 1A. Step 806 can also include acquiring one or more reflected light images based on visible light reflected from the anatomy of interest, and/or one or more fluorescence images based on fluorescence emitted by the fluorescence imaging agent excited by the fluorescence excitation light, such as via image acquisition assembly 20 of FIG. 1A. Step 806 can also include generating one or more color images in which fluorescence signal from the fluorescence imaging agent is represented in one of the constituent colors (e.g., green) of the one or more color images. Step 806 can also include displaying one or more color images to a user.

At step 808, one or more color images generated in step 806 are stored in one or more memories, which can include a memory of the surgical visualization system, a portable memory device, a networked memory location (e.g., cloud storage), and/or any other suitable memory. Step 808 include storing one or more color images in which fluorescence of a fluorescence imaging agent is represented in one of the constituent colors of the color image. Step 808 may also include storing one or more color images that do not include contribution from fluorescence of a fluorescence imaging agent, such as a color image acquired when there was not fluorescence imaging agent present in the anatomy of interest, when no fluorescence excitation light was illuminating the anatomy of interest, or a color image generated without using an acquired fluorescence signal.

Method 800 may include optional step 810 in which one or more new values for one or more parameters of the study are selected according to the study goals. For example, a different fluorescence imaging agent may be selected for a study evaluating different imaging agents or a different dose may be selected for a study determining an optimal or near-optimal dose. Where the study is for comparing different image-guided surgical systems, a different image-guided surgical system may be selected. Where the study is for comparing different settings of an image-guided surgery, one or more new settings may be selected. The one or more settings could include, for example, an amount of fluorescence excitation light, an amount or color temperature of white light, an exposure setting of an image sensor, and/or one or more parameters used in an algorithm for generating a color image from white light and fluorescence light images. Steps 804 to 808 may then be repeated (e.g., on a different subject) with the new selection(s). For studies that involve evaluating different surgical visualization systems or different settings for a surgical visualization system, step 804 may be skipped and the different surgical visualization system or different settings for surgical visualization system may be used for generating color images from the same subject

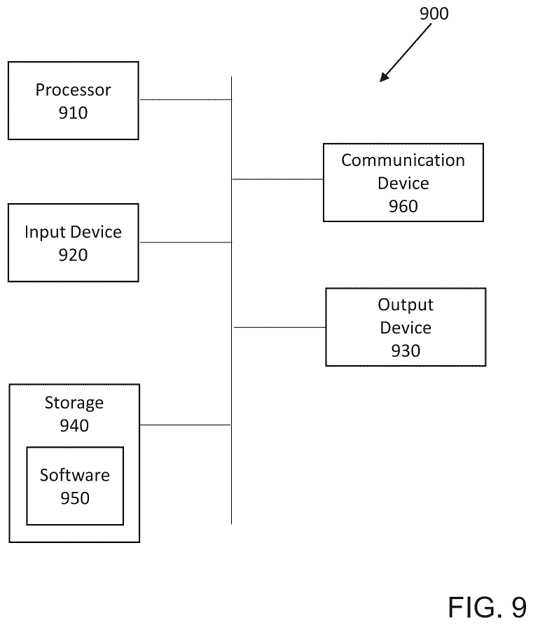

At step 812, one or more color images stored in step 808 are received by a computing system, such as computing system 900 of FIG. 9, from the memory where the one or more color images were stored (which can be a memory of the computing system or a memory of another computing system communicatively coupled to the computing system). At step 814, one or more measures of contrast enhancement is generated, according to method 100, based on the one or more color images received at step 812.

At step 816, the one or more measures of contrast enhancement are used for evaluation of one or more aspects of the study, such as for evaluating the one or more selections made at step 802. For example, a measure of contrast enhancement can be used as an absolute quantification of a surgeon's observed visualization of the anatomy of interest (e.g., as compared to one or more established thresholds or ranges of values of measures) or as a relative quantification that compares different selections made at step 802 and 810. The measure of contrast enhancement can be used to determine the near optimal or optimal dose of the fluorescence imaging agent, such as by comparing a measure of contrast enhancement generated for a first dose set at step 804 and a second dose set at step 810. Method 800 may include using the determination of the near optimal or optimal dose in preparing fluorescence imaging agent for use, such as in packaging fluorescence imaging agent for distribution to third parties for administration to patients. This can include packaging fluorescence imaging agent in one or more quantities that are based on the determined optimal or near optimal dose. For example, where an optimal dose is determined to be 1.0 mg, the fluorescence imaging agent may be packages in units of 1.0 mg. The measure of contrast enhancement can be used as a biomarker of a surgeon's ability to visualize and/or identify the anatomy of interest. For example, the measure of contrast enhancement can be compared to an establish baseline of values for the measure of contrast enhancement to determine the surgeon's ability to visualize and/or identify the anatomy of interest. Measures of contrast enhancement can be compared to evaluate different surgical visualization systems or different settings for a surgical visualization system—i.e., determining whether a given system or set of system settings showed a statistically significant improvement over another system or set of system settings. As such, method 800 may include configuring a surgical visualization system based on a comparison of on one or more measures of contrast enhancement. For example, a high measure of contrast enhancement may be associated with particular surgical visualization system settings, and a surgical visualization system may be configured according to the particular surgical visualization system settings for a surgical visualization session. This could include, for example, a setting for intensity of a fluorescence excitation and/or a gain setting for an imaging sensor.

Step 816 can include using the one or more measures of contrast enhancement for determining a relationship between a fluorescence imaging agent-based visual enhancement in a color surgical video image of a patient and the concentration of the fluorescence imaging agent in a biological sample from the patient. This can include calculating the relationship between the one or more measures of contrast enhancement and the concentration of the fluorescence imaging agent in the biological sample. Optionally, a measure of visual enhancement can be determined, at step 814, for each of two or more different dosage amounts of the fluorescence imaging agent, and the relationship between the respective measure of contrast enhancement and the concentration of the fluorescence imaging agent in the biological sample at each of the two or more dosage amounts can be calculated. Determining the relationship between a fluorescence imaging agent-based visual enhancement in a color surgical video image of a patient and the concentration of the fluorescence imaging agent in a biological sample from the patient can include determining a dosage range over which there is a positive linear correlation between the fluorescence imaging agent-based visual enhancement in the color surgical video image of the patient and the concentration of the fluorescence imaging agent in the biological sample from the patient. The biological sample can include, for example, urine, blood, lymphatic fluid, or feces.

The measure of contrast enhancement (also referred to as a visual enhancement value) may be and/or indicate a numerical value representing the visibility improvement of the anatomy or pathology of interest based on the presence of the fluorescence imaging agent. For example, the measure of contrast enhancement may be 30%, indicating that the presence of the fluorescence imaging agent improved the visibility of the anatomy of pathology of interest by 30% relative to the portions of the anatomy or pathology of interest that do not contain any fluorescence imaging agent. As another example, the measure of contrast enhancement may be a single numerical or alphanumerical value representing a general visibility improvement of the first region of the anatomy or pathology of interest. In particular, the measure of contrast enhancement may be included as part of a three-point visual enhancement scale (e.g., a three-point Likert scale) indicating a visibility improvement of the first region of the anatomy or pathology of interest based on presence of the fluorescence imaging agent. In some aspects, the measure of contrast enhancement may be included as part of a five-point visual enhancement scale (e.g., a five-point Likert scale) indicating a visibility improvement of the first region of the anatomy or pathology of interest based on presence of the fluorescence imaging agent. In clinical terms, the measure of contrast enhancement can be a measure of the perceived conspicuity of the target anatomy or pathology characterized by the proportion of fluorescent light signal to reflected light signal or characterized as the identification of the target anatomy or pathology location.

Method 800 or one or more methods performed subsequent to method 800 can include using one or more measures of contrast enhancement for determining and/or selecting an optimal or near optimal fluorescence imaging agent dose, an optimal or near optimal fluorescence imaging agent type, an optimal or near optimal visualization system, and/or optimal or near optimal visualization system settings. Any such method may include using the optimal or near optimal fluorescence imaging agent dose, the optimal or near optimal fluorescence imaging agent type, the optimal or near optimal visualization system, and/or the optimal or near optimal visualization system settings while capturing one or more images of a scene of a patient that includes a fluorescence signal, e.g. at times when the fluorescence imaging agent is present (e.g. pre-administered) in the anatomy or pathology of interest and/or the anatomy or pathology of interest is illuminated with fluorescence excitation illumination. Any such method may include displaying the one or more images and/or generating a visualization based on the one or more images. For example, one or more fluorescence images of the scene may be displayed or may be used to generate a combined visible light and fluorescence image (e.g., overlay) of the anatomy of pathology of interest. Any such method may include analyzing one or more of the captured images to generate a quantification associated with the anatomy of pathology of interest. For example, a quantification of perfusion of the tissue by blood may be generated based on analysis of one or more of the captured images. For example, the relative intensities of different pixels may be used by a computing system to automatically generate a quantification of the amount of blood in different regions of the anatomy of pathology of interest. Optionally, a measure of contrast enhancement can be used in generating the quantification. For example, the measure of contrast enhancement may be used to normalize the quantification. A such, one or more methods may include, at a computing system, receiving one or more images of anatomy or pathology of interest captured while using the optimal or near optimal fluorescence imaging agent dose, the optimal or near optimal fluorescence imaging agent type, the optimal or near optimal visualization system, and/or the optimal or near optimal visualization system settings selected based on one or more measures of contrast enhancement, and based on the analysis, and automatically analyzing the one or more images to generate a quantification associated with the anatomy or pathology of interest, where the quantification is based on the one or more measures of contrast enhancement. Any of these methods can include generating and displaying a visualization based on the quantification. For example, a visualization can be displayed that overlays a heat map of tissue perfusion on a white light image of the anatomy of pathology of interest.

### Example Contrast Quantification for Selecting Fluorescence Imaging Agent Dose

The following example illustrates using contrast quantification, according to method 100, to assess the visual effectiveness of different fluorescence imaging agent doses. In this example, subjects undergoing laparoscopic/minimally invasive colorectal surgery received doses of either 0.3 mg, 1.0 mg, or 3 mg of pudexacianinium chloride (Chemical formula (A))—an indocyanine-green derivative fluorescence imaging agent with hydrophilic properties and rapid urinary clearance without metabolism after intravenous administration—at the start of the minimally invasive surgery procedure and the normal colectomy was completed.

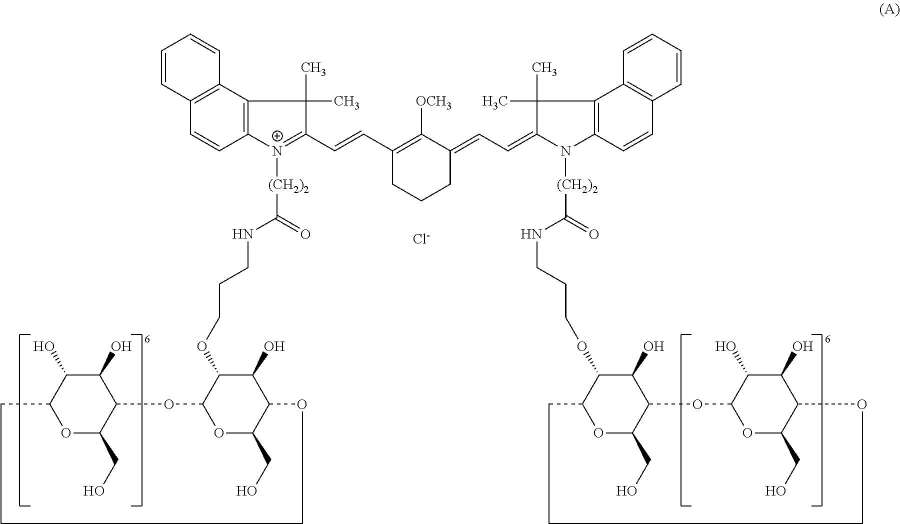

At pre-specified time intervals during the surgery, the surgeon utilized a fluorescence visualization system to examine the retroperitoneum and attempted to visualize the ureters. The surgical videos generated by the fluorescence visualization system were recorded. Frames from these surgical videos were analyzed according to method 100 to quantify the color contrast observable by the surgeon when a bolus of the fluorescence imaging agent passed through the ureters.

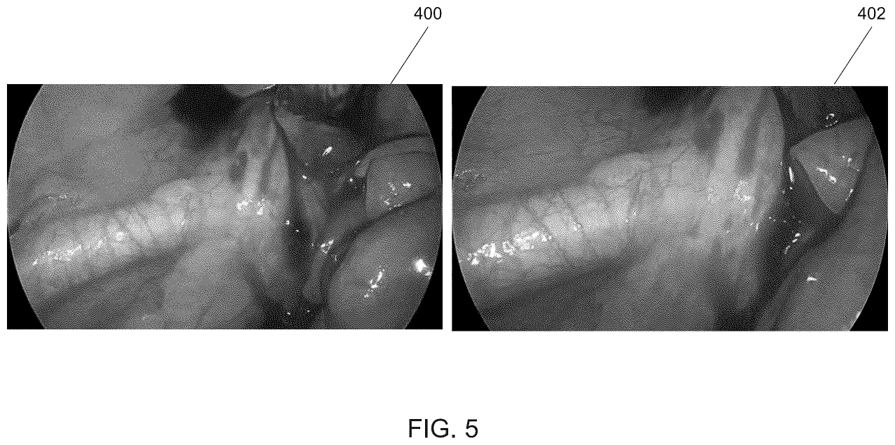

Baseline and fluorescence-containing images for a subject who received a 1.0 mg dose of the fluorescence imaging agent are illustrated in FIG. 5—baseline image 400 and fluorescence-containing image 402. The color images 400, 402 are full color surgical video frames generated by a fluorescence visualization system that combines full color and fluorescence frames into a combined frame in which the fluorescence signal is captured in the green component of the frame. The color images 400, 402 capture a portion of a ureter, which is visible in bright green in fluorescence-containing image 402. The fluorescence-containing image 402 was captured while a bolus of the fluorescence imaging agent was present in the portion of the ureter within the field of view, and the baseline image 400 was captured shortly after the bolus of the fluorescence imaging agent had at least partially passed through the portion of the ureter.

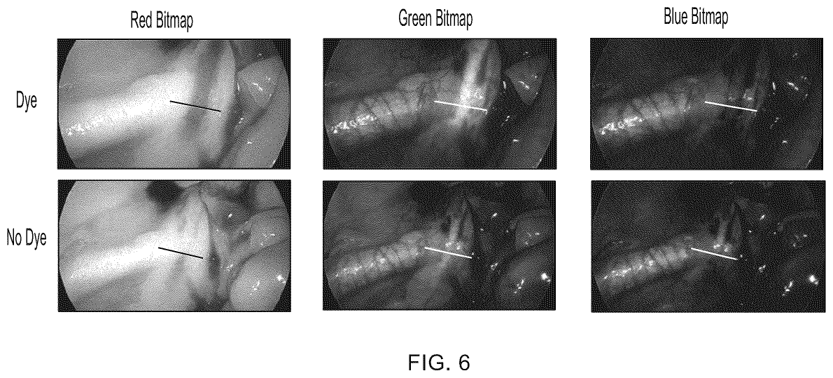

Each of the color images 400, 402 was imported in a video analysis software and exported as separate greyscale bitmaps of the red (R), green (G) and blue (B) color components corresponding to each full color image frame. FIG. 6 shows the bitmaps for each color component for each of the fluorescence imaging agent and baseline images.

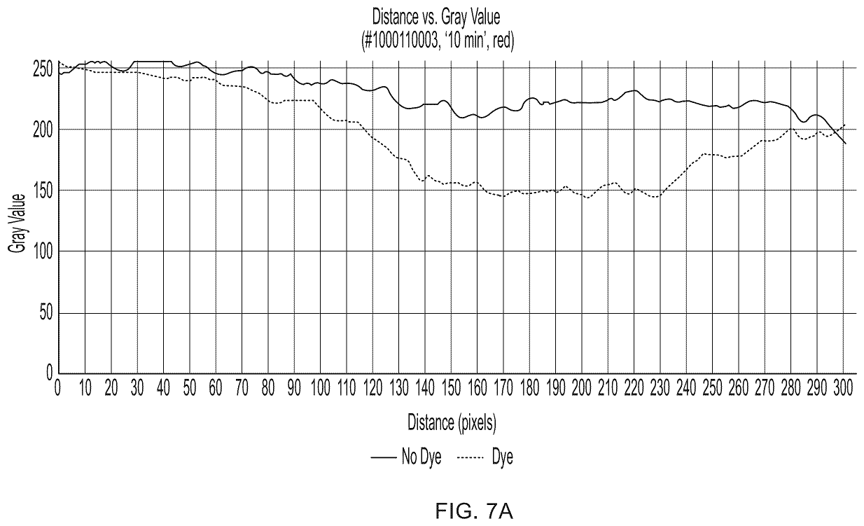

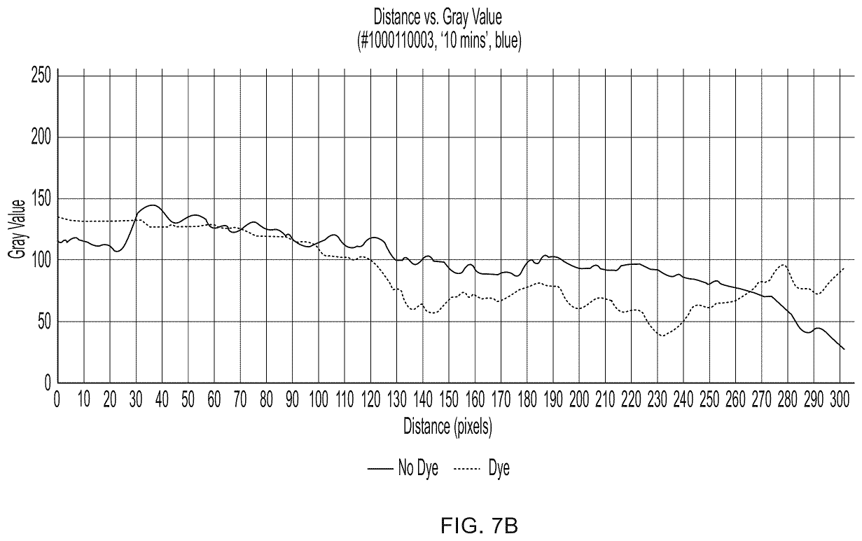

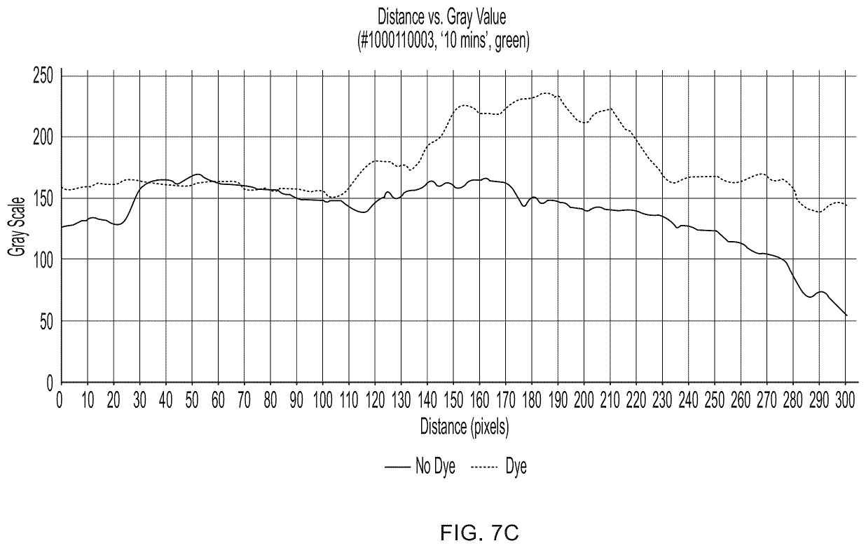

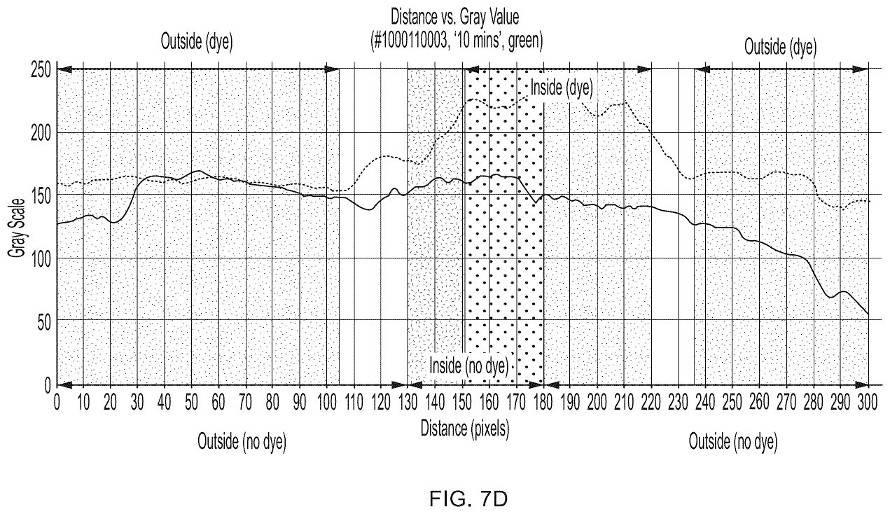

Next, a line was plotted that transects the ureter at the same location on both the fluorescence-containing and baseline bitmaps for each R, G, and B color component. Graphs were subsequently created that plot the bitmap signal intensity values at each point along the line segment against position on the line segment for each line segment in each color component. Red, green and blue graphs were constructed for each pair of fluorescence-containing — baseline color components. Exemplary graphs are illustrated in FIGS. 7A-C, which include the graphs for each color component for the fluorescence-containing image (“dye”) and baseline image (“no-dye”) on the same plot. FIG. 7A is the graph for the red color component for the fluorescence-containing and baseline images, FIG. 7B is the graph for the blue color component for the fluorescence-containing and baseline images, and FIG. 7C is the graph for the green color component for the fluorescence-containing and baseline images,

At least the graph for the green component was then analyzed to identify, according to steps 104 and 106 of method 100, regions along the line segment (shown in FIG. 7D as position 0-300) that—by comparison with the bitmap—lie clearly inside the anatomical boundaries of the ureter (a region encompassed by the anatomy of interest according to step 104) or clearly outside of those same anatomical boundaries (a region not encompassed by the anatomy of interest according to step 106). Note that the “inside” and “outside” regions are different for the fluorescence imaging agent (“dye”) and baseline (“no Dye”) images due to the differences in the field of view of the color images.

The signal values over those “inside” and “outside” regions of the color image were then averaged and tabulated as shown in the table below (the table show results for all three color components in the fluorescence imaging agent and baseline frames.)

| Dye | | | | | | No Dye | | | | | |
|---|---|---|---|---|---|---|---|---|---|---|---|
| R inside | R outside | G inside | G outside | B inside | B outside | R inside | R outside | G inside | G outside | B inside | B outside |
| 150.033 | 211.194 | 223.920 | 159.110 | 70.677 | 98.967 | 216.035 | 232.233 | 159.190 | 134.211 | 93.908 | 100.268 |

Thus, for example, for the green component for the fluorescence-containing image, the average of the intensity values for the pixels of the inside region was 223.920 and the average of the intensity values for the pixels of the outside region was 159.110.

As noted above, the fluorescence emitted by the fluorescence imaging agent shown using the green component in the surgical video. A relative contribution of the green signal value to the blue and red signal values was computed for the inside and outside regions using the following equation:

$$\\ relative contribution = ( \frac{G}{R + B} )$$

where R, G, and B are the average values for each of the color components in the respective regions. As some color contrast between the ureter and the surrounding tissue may exist naturally, the degree to which this contrast is enhanced by the presence of the fluorescence imaging agent in the ureter was quantified by calculating a ratio between the relative contributions of the green signal for the fluorescence imaging agent for the inside and outside regions, as follows:

$$\\ Contrast Quantification = \frac{{( \frac{G}{R + B} )}_{Inside}}{{( \frac{G}{R + B} )}_{Outside}}$$

Using the values from the above table, the contrast quantification for the fluorescence imaging agent and baseline images are: 2.0 and 1.3, respectively.

Contrast quantification values were calculated, according to the above process, for fluorescence-containing and baseline image pairs for each subject. The results are tabulated in the below table.

| | ALL PATIENTS | | | | | |
|---|---|---|---|---|---|---|
| | 0.3 mg | | 1.0 mg | | 3.0 mg | |
| | Fluorescence- | Baseline | Fluorescence- | Fluorescence- | Fluorescence- | Fluorescence- |
| | containing image | image | containing image | containing image | containing image | containing image |
| N | 3 | | 6 | | 3 | |
| Mean | 1.84 | 1.10 | 2.55 | 1.06 | 3.23 | 1.09 |
| Standard Deviation | 0.46 | 0.13 | 0.95 | 0.12 | 1.70 | 0.10 |
| Median | 1.77 | 1.05 | 2.28 | 1.06 | 2.64 | 1.10 |

Quantifying the color contrast observed by the surgeon when a bolus of the fluorescence imaging agent passes through the ureters by calculating a contrast quantification, as described above, enables an assessment of how such contrast varies with dose of the fluorescence imaging agent. The quantification results can provide additional evidence supporting the selection of the appropriate dose of the fluorescence imaging agent.

As shown in the above table, the calculated contrast quantification is notably less for the 0.3 mg dose of the fluorescence imaging agent than for the higher (1.0 mg and 3.0 mg) doses of the fluorescence imaging agent. In that regard, the higher doses can be considered as providing a greater assurance of enabling the surgeon to visualize the ureter consistently and easily. There is an apparent “leveling off” in contrast enhancement at the higher doses, with the mean contrast quantification being quantitatively similar—albeit slightly lower for the 1.0 mg dose.

The fact that—in the absence of the fluorescence imaging agent bolus—ureters are generally difficult for the surgeon to distinguish from the surrounding tissues and the contrast quantification value for the baseline images (the baseline) was around 1 demonstrates that the contrast quantification is representative of the color contrast observed by the surgeon.

### Example of Using a Contrast Enhancement Factor as an Alternative to Biological Samples for Bioequivalence & Bioavailability Assessments

The following example illustrates using a measure of visual enhancement of the anatomy of interest in a color image (a contrast enhancement factor), generated according to method 100, as an alternative to biological samples for bioequivalence and bioavailability assessments of a fluorescence imaging agent, in particular the fluorescence dye ASP5354 (pudexacianinium). In this example, two in vitro studies were performed to determine the effect of ASP5354 concentration on near-infrared fluorescence (NIR-F). In the first study, ASP5354 (0.01-1000 μg/mL) was added to 96 well plates, the plates placed in a microplate reader, and subjected to a near-infrared excitation frequency of 780 nm. The emitted NIR-F determined at a wavelength of 820 nm. The fluorescence intensity increased as the concentration of pudexacianinium increased and tended to plateau at concentrations greater than or equal to 100 μg/mL.

In the second study, pudexacianinium (0.01-10 μg/mL) was instilled in pig ureters ex vivo and the NIR-F determined using a PDE-NEO NIR-F imager. Here too, the NIR-F intensity increased with increasing concentrations of pudexacianinium with the fluorescence intensity tending to plateau at concentrations equal to or greater than 1 μg/mL, as shown in the table below. Thus, the fluorescence activity of pudexacianinium can be visualized in the lumen of ureters.

| | Pudexacianinium Concentration (ug/mL) | | | | | | | |
|---|---|---|---|---|---|---|---|---|
| | 0 | 0.01 | 0.03 | 0.1 | 0.3 | 1.0 | 3.0 | 10 |
| NIR-F | 0.000 | 0.000 | 4.672 | 47.945 | 125.078 | 229.504 | 254.867 | 254.808 |
| Intensity | (0.000) | (0.000) | (6.183) | (18.826) | (35.574) | (28.243) | (0.065) | (0.295) |

NIR-F: near-infrared fluorescence
NIR-F is expressed in arbitrary units as mean (SD) for three ureters per concentration.

Next, the ability to visualize ureters in vivo was assessed in Gottingen minipigs. The animals were anesthetized, laparotomized and then intravenously injected with pudexacianinium (0.001 or 0.01 mg/kg). Videos of the abdominal cavity were taken using a NIR-F camera and assessed visually by three independent clinicians for up to three hours. At the low dose (0.001 mg/kg), only one out of three animals demonstrated ureteral visualization for three hours. However, at the high dose level (0.01 mg/kg), three out of three animals showed ureteral visualization for the full three hour observation period.

In a second qualitative in vivo study, pudexacianinium (0.01 mg/kg, iv) was injected into a healthy female Yorkshire pig and the ureters were visualized using examples of an image guided surgery system 10 of FIG. 1A—a Stryker Corporation 1588 AIM System and a Stryker Corporation SPY-PHI open field system—prior to any dissection. Using either system, the ureters were readily visible to the investigators indicating that different near infrared imaging systems can be used to visualize the ureters following intravenous pudexacianinium administration.

Five important conclusions can be drawn from these preclinical studies: 1) ASP5354 can emit in the NIR-F range following excitation at 780 nm, 2) the NIR-F emission observed was concentration dependent, 3) ASP5354 NIR-F emission was visible through ureteral tissue both in vitro and in vivo, 4) intravenous administration of ASP5354 at a dose of 0.01 mg/kg to minipigs resulted in visualization of the ureter for up to 3 hours, and 5) ASP5354 NIR-F fluorescence could be detected using different near infrared imaging systems.

ASP5354 at a dose of 0.01 mg/kg showed ureteral fluorescence that was subjectively determined to be adequate for ureteral visualization for a surgery period of 3 hours. Urine samples collected from these animals showed a concentration of pudexacianinium of 1-6 μg/mL over this same observation period. A direct translation of the nonclinical pharmacology visualization results in the intended clinical use and dose in humans is considered feasible given the simple elimination route of the dye in both cases (eliminated entirely through renal system) and given the physiologic comparability between porcine and human kidneys. The 0.01 mg/kg dose used in pigs translates to an approximate human equivalent dose of 0.7-0.8 mg/participant. This is consistent with the dose recommendation for the clinical Phase 3 study (1 mg/participant).

The pharmacokinetic modeling and simulation results based on the Phase 1 clinical study data suggest that a fixed dose of 1 mg would achieve an exposure of at least 1 μg/mL urine concentration comparable to the nonclinical pharmacology model, for approximately 3 hours post administration. The results from the Phase 1 study provided the foundation for Phase 2 study, a randomized open-label, dose ranging study for ureter visualization, using ASP5354 in participants undergoing laparoscopic/minimally invasive colorectal surgery.

The Phase 2 results were reviewed by a Visualization Review Committee (VRC) to determine (1) if ureter visualization was achieved at 30 minutes and at end of surgery for a given dose evaluated; and (2) the dose. The VRC concluded that pudexacianinium was shown to provide enhanced visualization of ureter(s) under NIR-F conditions at all doses; however, visualization throughout the duration of the surgical procedure consistently occurred at a single dose of 1 mg and 3 mg per participant. The lower dose (0.3 mg per participant) was determined to be inadequate for ureter(s) visualization due to a lower fluorescence intensity and shorter duration of visualization than doses of 1 mg and 3 mg per participant. The VRC indicated that the 1 mg per participant dose provided ureter(s) visualization during the surgical procedure, but importantly at the beginning, and at the end. The results of the Phase 2 study support the use of pudexacianinium as an intraoperative ureter visualization (surgeon's ability to identify/locate the ureter from the surrounding structures) during the entire abdominopelvic surgical procedure. The VRC indicated that there were no notable differences in ureter(s) visualization between the 1 mg and 3 mg dose. The VRC stated that the stronger intensity seen in the 3 mg dose per participant did not provide a significant advantage over the 1 mg dose per participant for the surgeon's ability to visualize the ureter(s) during the entire surgical procedure. As a result, the VRC recommended moving forward with the 1 mg per participant dose for Phase 3.

In order to anchor the surgeons' (study investigators') pudexacianinium Phase 2 qualitative assessment to a measurable parameter, such as urine concentration, pudexacianinium spot urine concentrations were measured. However, due to the variability of spot urine concentration from participants undergoing surgery and the limited number of spot urine samples collected, it was difficult to anchor the urine concentration to the surgeons' qualitative assessment. The pre-specified Phase 2 exploratory variable, signal-to-background ratio (SBR) was not an appropriate indicator to quantify the intensity of signals because there were many factors potentially having an impact on the intensity of signals. In addition, surgeons often use a color overlay mode of a surgical visualization system when the function is available while SBR is obtained on black/white mode. Therefore, the use of a contrast enhancement factor (CEF), generating according to method 100, as an alternative to urine concentration as a quantitative marker was explored.

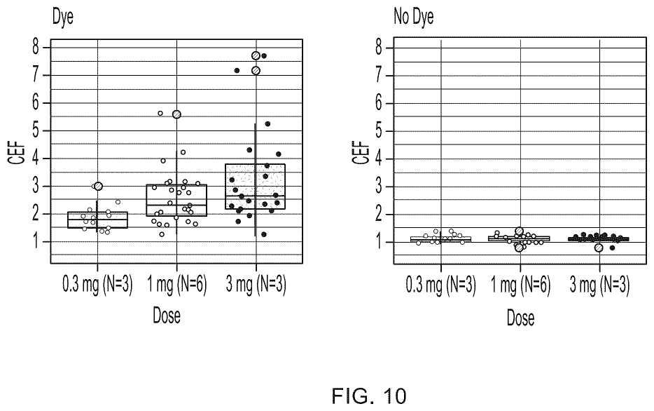

Summary statistics of the CEF factor for the Phase 2 video data are presented in the table below and in FIG. 10, which shows box plots of the CEF throughout time points.

| Presence of | | | | 1 mg (N = 6) | |
|---|---|---|---|---|---|
| Pudexacianinium | Summary | 0.3 mg | 1 mg | including | 3 mg |
| in Ureter | Statistics | (N = 3) | (N = 3) | expansion | (N = 3) |
| Dye | n/N | 14/3 | 15/3 | 27/6 | 21/3 |
| | Mean (SD) | 1.84 (0.46) | 2.97 (1.03) | 2.55 (0.95) | 3.23 (1.70) |
| | Median | 1.77 | 2.96 | 2.28 | 2.64 |
| | Min-Max | 1.3-3.0 | 1.6-5.6 | 1.3-5.6 | 1.2-7.7 |
| No Dye | n/N | 14/3 | 15/3 | 27/6 | 21/3 |
| | Mean (SD) | 1.10 (0.13) | 1.06 (0.12) | 1.06 (0.12) | 1.09 (0.10) |
| | Median | 1.05 | 1.07 | 1.06 | 1.10 |
| | Min-Max | 0.9-1.3 | 0.8-1.3 | 0.8-1.4 | 0.7-1.2 |

CEF: contrast enhancement factor
N and n represent number of participants and number of data, respectively.

The assessment shows stable reference CEF of almost 1 with low variability when no dye is present in the ureter. To evaluate contrast enhancement as a function of dose of pudexacianinium, the mean and standard deviation of the CEF values were calculated for each dose. Median and range of CEF at 0.3 mg was lower than 1 mg and 3 mg, while overlapped distribution of CEF with similar median values was observed between 1 mg and 3 mg in the image with dye. The results of the CEF for the first 9 patients assessed by the VRC were consistent with the surgeons' qualitative assessment that there were no clinically meaningful differences in ureter(s) visualization between the 1 mg and 3 mg dose. For all 12 patients, a correlation between the surgeon's assessment and the quantitative assessment was observed. Thus, CEF serves as a suitable, measurable parameter (quantitative assessment) to anchor the surgeon's qualitative assessment.

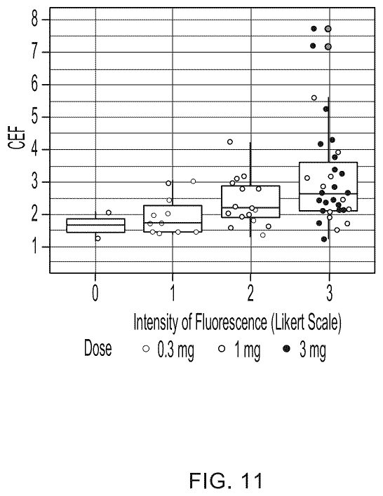

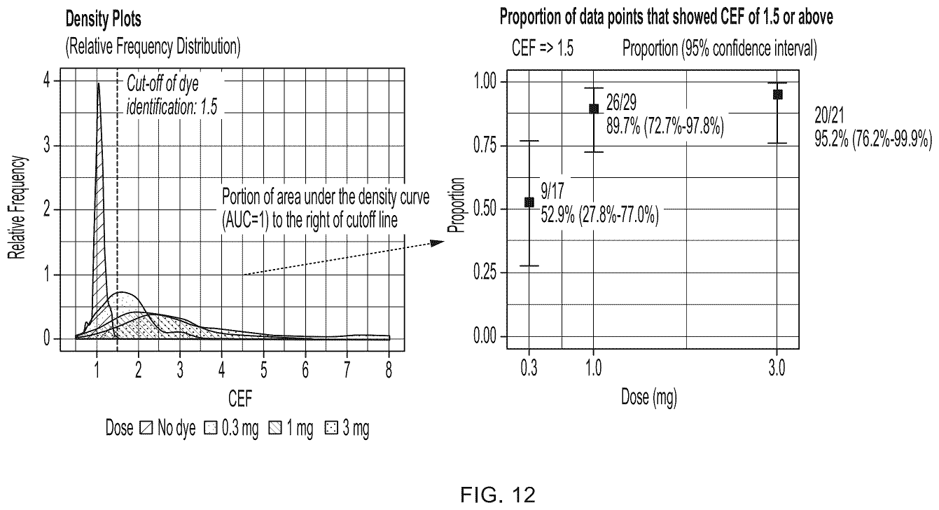

As shown by this example, the CEF is a quantitative measure that can be used to support or substitute the qualitative surgeons' assessment, as illustrated in FIG. 11. The quantitative assessment shows stable reference CEF value of almost 1 with low variability when no dye is present in the ureter (FIG. 12). All data points under no dye conditions showed CEF value of below 1.5. Therefore, cut-off (threshold) value for dye identification is set at 1.5. The data suggest that the 1.5 cut off is relevant because CEF above 1.5 suggest ureter identification/visualization. For 1 mg and 3 mg, the proportion of data above 1.5 is 90% or more (FIG. 12), and the 95% confidence interval almost overlapped between 1 mg and 3 mg, supporting the sponsor selected dose of 1 mg.

Simply put, a cut-off value for dye identification of 1.5 represents the CEF value under/over which a surgeon is able to visually discern the presence of a dye in an anatomy or pathology of interest. Any region of the anatomy or pathology of interest having a CEF value below 1.5 does not display sufficient visual contrast of a dye relative to the surrounding anatomy to be utilized by a surgeon during an operative procedure to locate the anatomy containing the dye. By contrast, any region of the anatomy or pathology of interest having a CEF value at or above 1.5 does display sufficient visual contrast of a dye relative to the surrounding anatomy to be utilized by a surgeon during an operative procedure to locate the anatomy containing the dye. In certain aspects, the CEF value and the corresponding cut-off value may be calculated and/or adjusted automatically by systems described herein, such as system 10 in combination with the external computing systems 52 of FIG. 1A.

### Example Computing System

FIG. 9 illustrates an example of a computing system 900 that can be used for performing one or more steps or one or more portions of one or more steps of method 100 for assessing fluorescence imaging agent-based visual enhancement in a color image or video frame generated by a surgical visualization system, such as system 10 of FIG. 1A. System 900 can be used, for example, for external computing systems 52 of FIG. 1A for performing one or more steps of method 100. System 900 can be a computer connected to one or more networks. System 900 may be communicatively connected via the one or more networks to an image guided surgery system, such as surgical visualization system 10 of FIG. 1A, and/or to one or more data storage system storing color images generated and stored by the image guided surgery system. System 900 can be a client or a server. As shown in FIG. 9, system 900 can be any suitable type of processor-based system, such as a personal computer, workstation, server, handheld computing device (portable electronic device) such as a phone or tablet, or dedicated device. The system 900 can include, for example, one or more of input device 920, output device 930, one or more processors 910, storage 940, and communication device 960. Input device 920 and output device 930 can generally correspond to those described above and can either be connectable or integrated with the computer.

Input device 920 can be any suitable device that provides input, such as a touch screen, keyboard or keypad, mouse, gesture recognition component of a virtual/augmented reality system, or voice-recognition device. The input device 920 can be used, for example, for receiving a user input corresponding with a selection of one or more images displayed by an output device 930 and/or one or more regions of an image displayed by an output device 930, according to one or more aspects of steps 102-106 of method 100. Output device 930 can be or include any suitable device that provides output, such as a display, touch screen, haptics device, virtual/augmented reality display, or speaker.

Storage 940 can be any suitable device that provides storage, such as an electrical, magnetic, or optical memory including a RAM, cache, hard drive, removable storage disk, or other non-transitory computer readable medium. Communication device 960 can include any suitable device capable of transmitting and receiving signals over a network, such as a network interface chip or device. The components of the computing system 900 can be connected in any suitable manner, such as via a physical bus or wirelessly.

Processor(s) 910 can be any suitable processor or combination of processors, including any of, or any combination of, a central processing unit (CPU), graphics processing unit (GPU), field programmable gate array (FPGA), and application-specific integrated circuit (ASIC). Software 950, which can be stored in storage 940 and executed by one or more processors 910, can include, for example, the programming that embodies the functionality or portions of the functionality of the present disclosure (e.g., as embodied in the devices as described above). For example, software 950 can include one or more programs for execution by one or more processor(s) 910 for performing one or more of the steps of method 100 or portions of one or more steps of method 100.

Software 950 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 940, that can contain or store programming for use by or in connection with an instruction execution system, apparatus, or device.

Software 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport computer readable medium can include, but is not limited to, an electronic, magnetic, optical, electromagnetic, or infrared wired or wireless propagation medium.

System 900 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 900 can implement any operating system suitable for operating on the network. Software 950 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

### Examples of Suitable Fluorescence Imaging Agents

The methods described above can be used for quantifying contrast for imaging associated with any suitable fluorescence imaging agent (also referred to herein interchangeably as optical agent). As noted in the example above, a fluorescence imaging agent can be pudexacianinium chloride. The following provides examples of other fluorescence imaging agents that may be used.

The fluorescence imaging agent or optical agent may be a non-toxic imaging agent which fluoresces when exposed to radiant energy, e.g., light. The fluorescence imaging agent may be a fluorescence imaging agent that emits light in the infrared spectrum. The fluorescence imaging agent or optical agent may be selected from the group consisting of phenylxanthenes, phenothiazines, phenoselenazines, cyanines, indocyanines, squaraines, dipyrrolo pyrimidones, anthraquinones, tetracenes, quinolines, pyrazines, acridines, acridones, phenanthridines, azo fluorescence imaging agents, rhodamines, phenoxazines, azulenes, azaazulenes, triphenyl methane fluorescence imaging agents, indoles, benzoindoles, indocarbocyanines, benzoindocarbocyanines, and BODIPY™ derivatives having the general structure of 4,4-difluoro-4-bora-3a,4a-diaza-s-indacene. Specific optical agents that may be used in the process include, but are not limited to, for example, indocyanine green, indocyanine green conjugates, indocyanine-dodecaaspartic acid conjugates, and indocyanine (NIRD)-polyaspartic acid conjugates fluorescein, fluorescein isothiocyanate, fluorescein-polyaspartic acid conjugates, fluorescein-polyglutamic acid conjugates, fluorescein-polyarginine conjugates, rhodamine, phycoerythrin, phycocyanin, allophycocyanin, o-phthaldehyde, fluorescamine, Rose Bengal, trypan blue, methylene blue, methylene blue conjugates, fluoro-gold, isosulfan blue, indole disulfonates, benzoindole disulfonate, bis(ethylcarboxymethyl)indocyanine, bis(pentylcarboxymethyl)indocyanine, polyhydroxyindole sulfonates, polyhydroxybenzoindole sulfonate, rigid heteroatomic indole sulfonate, indocyaninebispropanoic acid, indocyaninebishexanoic acid, 3,6-dicyano-2,5-[(N,N,N′,N′-tetrakis(carboxymethyl)amino]pyrazine, 3,6-[(N,N,N′,N′-tetrakis(2-hydroxyethyl)amino]pyrazine-2,5-dicarboxylic acid, 3,6-bis(N-azatedino)pyrazine-2,5-dicarboxylic acid, 3,6-bis(N-morpholino)pyrazine-2,5-dicarboxylic acid, 3,6-bis(N-piperazino)pyrazine-2,5-dicarboxylic acid, 3,6-bis(N-thiomorpholino)pyrazine-2,5-dicarboxylic acid, 3,6-bis(N-thiomorpholino)pyrazine-2,5-dicarboxylic acid S-oxide, 2,5-dicyano-3,6-bis(N-thiomorpholino)pyrazine S,S-dioxide, indocarbocyaninetetrasulfonate, chloroindocarbocyanine, and 3,6-diaminopyrazine-2,5-dicarboxylic acid, and 3,6-diaminopyrazine-2,5-dicarboxylic acid.

The fluorescence imaging agent may be selected from the group consisting of fluorescein, indocyanine green, and methylene blue.

The fluorescence imaging agent may be administered at a dose of 0.3 mg, 1 mg, 3 mg, 10 mg, 25 mg, 50 mg, or 100 mg. The fluorescence imaging agent may be administered at a dose of 0.1 mg to 100 mg, 0.1 mg to 50 mg, 0.1 mg to 25 mg, 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, 0.1 mg to 0.5 mg, 0.5 mg to 5 mg, 0.5 mg to 4.5 mg, 0.5 mg to 4 mg, 0.5 mg to 3.5 mg, 0.5 mg to 3 mg, 0.5 mg to 2.5 mg, 0.5 mg to 2 mg, 0.5 mg to 1.5 mg, 0.5 mg to 1 mg, 1 mg to 5 mg, 1 mg to 4.5 mg, 1 mg to 4 mg, 1 mg to 3.5 mg, 1 mg to 3 mg, 1 mg to 2.5 mg, 1 mg to 2 mg, 1 mg to 1.5 mg, 2 mg to 5 mg, 2 mg to 4.5 mg, 2 mg to 4 mg, 2 mg to 3.5 mg, 2 mg to 3 mg, 2 mg to 2.5 mg, 2.5 mg to 5 mg, 2.5 mg to 4.5 mg, 2.5 mg to 4 mg, 2.5 mg to 3.5 mg, or 2.5 mg to 3 mg. 1 mg to 25 mg, 1 mg to 50 mg, 1 mg to 100 mg, 10 mg to 25 mg, 10 mg to 50 mg or 10 mg to 100 mg. It should be understood that the amount of fluorescence imaging agent to be administered may vary depending on the fluorescence imaging agent selected for administration.

The fluorescence imaging agent may be a phenylzanthene dye comprising the following chemical structure:

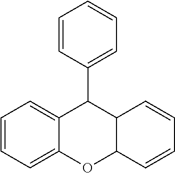 [SMILES: C1=CC2Oc3ccccc3C(c3ccccc3)C2C=C1]

wherein the structure is optionally substituted with one or more suitable chemical substituents. The phenylzanthene dye may be administered at a dose of 0.3 mg, 1 mg, or 3 mg. The phenylzanthene dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a phenothiazine dye comprising the following chemical structure:

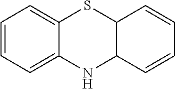 [SMILES: C1=CC2Nc3ccccc3SC2C=C1]

wherein the structure is optionally substituted with one or more suitable chemical substituents. The phenothiazine dye may be administered at a dose of 0.3 mg, 1 mg, or 3 mg. The phenothiazine dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a phenoselenazine dye comprising the following chemical structure:

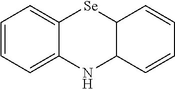 [SMILES: C1=CC2Nc3ccccc3[Se]C2C=C1]

wherein the structure is optionally substituted with one or more suitable chemical substituents. The phenoselenazine dye may be administered at a dose of 0.3 mg, 1 mg, or 3 mg. The phenoselenazine dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a cyanine dye comprising the following chemical structure:

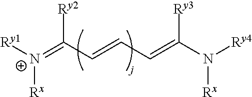

wherein R^{y1} and R^{y2} are each independently a suitable chemical substituent, or R^{y1} and R^{y2} are taken together to form an optionally substituted ring; R^{y3} and R^{y4} are each independently a suitable chemical substituent, or R^{y3} and R^{y4} are taken together to form an optionally substituted ring; each R^{x} is independently a suitable chemical substituent; and j is an integer, wherein the structure is optionally substituted with one or more suitable chemical substituents. The cyanine dye may be administered at a dose of 0.3 mg, 1 mg, or 3 mg. The cyanine dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a squaraine dye comprising the following chemical structure:

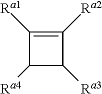

wherein R^{a1} and R^{a2} are selected from —R^{a5} and —O⁻, R^{a1} and R^{a2} are selected from ═R^{a6} and ═0, wherein R^{a5} and R^{a6} are each independently selected from any suitable chemical substituents, and the structure is optionally substituted with one or more suitable chemical substituents. The squaraine dye may be administered at a dose of 0.3 mg, 1 mg, or 3 mg. The squaraine dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be an anthraquinone dye comprising the following chemical structure:

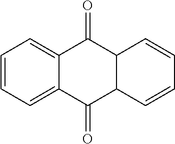 [SMILES: O=C1c2ccccc2C(=O)C2C=CC=CC12]

wherein the structure is optionally substituted with one or more suitable chemical substituents. The anthraquinone dye may be administered at a dose of 0.3 mg, 1 mg, or 3 mg. The anthraquinone dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a tetracene dye comprising the following chemical structure:

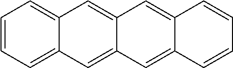 [SMILES: c1ccc2cc3cc4ccccc4cc3cc2c1]

wherein the structure is optionally substituted with one or more suitable chemical substituents. The tetracene dye may be administered at a dose of 0.3 mg, 1 mg, or 3 mg. The tetracene dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a quinoline dye comprising the following chemical structure:

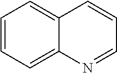

wherein the structure is optionally substituted with one or more suitable chemical substituents. The quinoline dye may be administered at a dose of 0.3 mg, 1 mg, or 3 mg. The quinoline dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a pyrazine dye comprising the following chemical structure:

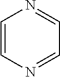

wherein the structure is optionally substituted with one or more suitable chemical substituents. The pyrazine dye may be administered at a dose of 0.3 mg, 1 mg, or 3 mg. The pyrazine dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be an acridine dye comprising the following chemical structure:

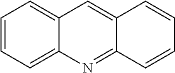 [SMILES: c1ccc2nc3ccccc3cc2c1]

wherein the structure is optionally substituted with one or more suitable chemical substituents. The acridine dye may be administered at a dose of 0.3 mg, 1 mg, or 3 mg. The acridine dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be an acridone dye comprising the following chemical structure:

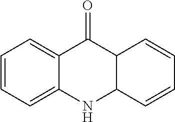 [SMILES: O=C1c2ccccc2NC2C=CC=CC12]

wherein the structure is optionally substituted with one or more suitable chemical substituents. The acridone dye may be administered at a dose of 0.3 mg, 1 mg, or 3 mg. The acridone dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a phenanthridine dye comprising the following chemical structure:

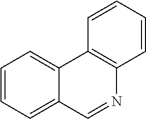 [SMILES: c1ccc2c(c1)cnc1ccccc12]

wherein the structure is optionally substituted with one or more suitable chemical substituents. The phenanthridine dye may be administered at a dose of 0.3 mg, 1 mg, or 3 mg. The phenanthridine dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be an azo dye comprising the following chemical structure:

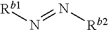

wherein R^{b1} and R^{b2} are each independently a suitable chemical substituent. The azo dye may be administered at a dose of 0.3 mg, 1 mg, or 3 mg. The azo dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a rhodamine dye comprising the following chemical structure:

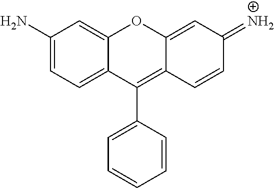 [SMILES: Nc1ccc2c(-c3ccccc3)c3ccc(=[NH2+])cc-3oc2c1]

wherein the structure is optionally substituted with one or more suitable chemical substituents. The rhodamine dye may be administered at a dose of 0.3 mg, 1 mg, or 3 mg. The rhodamine dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a phenoxazine dye comprising the following chemical structure:

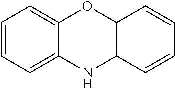 [SMILES: C1=CC2Nc3ccccc3OC2C=C1]

wherein the structure is optionally substituted with one or more suitable chemical substituents. The phenoxazine dye may be administered at a dose of 0.3 mg, 1 mg, or 3 mg. The phenoxazine dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be an azulene dye comprising the following chemical structure:

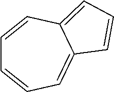

wherein the structure is optionally substituted with one or more suitable chemical substituents. The azulene dye may be administered at a dose of 0.3 mg, 1 mg, or 3 mg. The azulene dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be an azazulene dye comprising the following chemical structure:

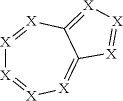

wherein each X is independently selected from CH and N, and wherein the structure is optionally substituted with one or more suitable chemical substituents. The azazulene dye may be administered at a dose of 0.3 mg, 1 mg, or 3 mg. The azazulene dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a triphenylmethane dye comprising the following chemical structure:

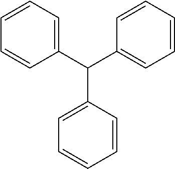 [SMILES: c1ccc(C(c2ccccc2)c2ccccc2)cc1]

wherein the structure is optionally substituted with one or more suitable chemical substituents. The triphenylmethane dye may be administered at a dose of 0.3 mg, 1 mg, or 3 mg. The triphenylmethane dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be an indole dye comprising the following chemical structure:

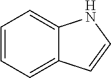

wherein the structure is optionally substituted with one or more suitable chemical substituents. The indole dye may be administered at a dose of 0.3 mg, 1 mg, or 3 mg. The indole dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a benzoindole dye comprising the following chemical structure:

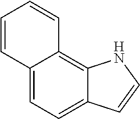 [SMILES: c1ccc2c(c1)ccc1cc[nH]c12]

wherein the structure is optionally substituted with one or more suitable chemical substituents. The benzoindole dye may be administered at a dose of 0.3 mg, 1 mg, or 3 mg. The benzoindole dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be an indocarbocyanine dye comprising the following chemical structure:

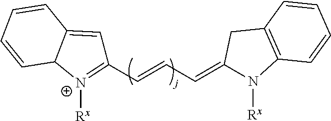

wherein each W is independently a suitable chemical substituent and j is an integer, and the structure is optionally substituted with one or more suitable chemical substituents. The indocarbocyanine dye may be administered at a dose of 0.3 mg, 1 mg, or 3 mg. The indocarbocyanine dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a benzoindocarbocyanine dye comprising the following chemical structure:

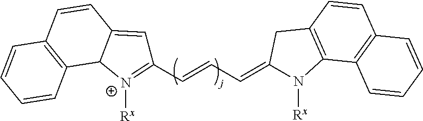

wherein each W is independently a suitable chemical substituent and j is an integer, and the structure is optionally substituted with one or more suitable chemical substituents. The benzoindocarbocyanine dye may be administered at a dose of 0.3 mg, 1 mg, or 3 mg. The benzoindocarbocyanine dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may have the general structure of 4,4-difluoro-4-bora-3a,4a-diaza-s-indacene as shown in the following chemical structure:

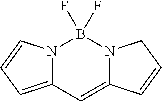

wherein the structure is optionally substituted with one or more suitable chemical substituents. The 4,4-difluoro-4-bora-3a,4a-diaza-s-indacene dye may be administered at a dose of 0.3 mg, 1 mg, or 3 mg. The 4,4-difluoro-4-bora-3a,4a-diaza-s-indacene dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

The fluorescence imaging agent may be a tricarbocyanine. The dye may be a tricarbocyanine dye having the general chemical formula shown below:

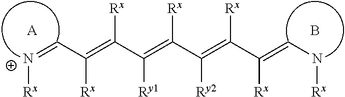

wherein A and B are optionally substituted rings; R^{y1} and R^{y2} are each independently a suitable chemical substituent, or R^{y1} and R^{y2} are taken together to form an optionally substituted ring; and each R^{x} is independently a suitable chemical substituent. The tricarbocyanine dye may be administered at a dose of 0.3 mg, 1 mg, or 3 mg. The tricarbocyanine dye may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg. The fluorescence imaging agent may be indocyanine green (ICG).

Fluorescence imaging agent analogs may be used. A fluorescence imaging agent analog includes a fluorescence imaging agent that has been chemically modified, but retains its ability to fluoresce when exposed to radiant energy of an appropriate wavelength. The fluorescence imaging agent may serve both an imaging function, as well as a therapeutic function.

A suitable fluorescence imaging agent may be a chemical compound having a structure corresponding to formula (1):

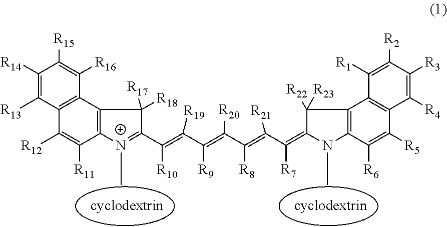

or a salt thereof, wherein R₁, R₂, R₃, R₄, R₅, R₆, R₇, R₁₀, R₁₁, R₁₂, R₁₃, R₁₄, R₁₅, R₁₆, R₁₇, R₁₈, R₁₉, R₂₀, R₂₁, R₂₂ and R₂₃ are each independently selected from the group consisting of hydrogen, alkyl, aryl, halogen, alkoxy, —NR^{a}R^{b}, —(NR^{a}R^{b}R^{c})⁺, carboxyl, formyl, sulfonyl, sulfonic acid, phosphate, alkyloxycarbonyl, aryloxycarbonyl, alkylcarbonyl, arylcarbonyl, and a heterocyclic ring; R₈ and R₉ are each independently selected from the group consisting of hydrogen, alkyl, aryl, halogen, alkoxyl, —NR^{a}R^{b}, —(NR^{a}R^{b}R^{c})⁺, carboxyl, formyl, sulfonyl, sulfonic acid, phosphate, alkyloxycarbonyl, aryloxycarbonyl, alkylcarbonyl, arylcarbonyl, and a heterocyclic ring, or R₈ and R₉ are taken together to form —CH₂—, —CH₂CH₂—, —CH₂CH₂CH₂— or —CH₂CH₂CH₂CH₂—; R^{a}, R^{b} and R^{c} are each independently selected from the group consisting of hydrogen and alkyl; and the alkyl group of R₁₋₂₃, R^{a}, R^{b} or R^{c} is optionally substituted with one or more substituents selected from the group consisting of alkyl, aryl, halogen, alkoxy, amino, carboxyl, formyl, sulfonyl, sulfonic acid, phosphate, alkyloxycarbonyl, aryloxycarbonyl, alkylcarbonyl, arylcarbonyl, and a heterocyclic ring. The fluorescence imaging agent may be a sodium salt, a potassium salt, or a magnesium salt of a chemical compound of formula (1). The fluorescence imaging agent may be a chloride salt of a chemical compound of formula (1).

The fluorescence imaging agent may be a cyclodextrin-bonded indocyanine compound in which at least a part of a naphthyl group of an indocyanine is included in a cavity of a cyclodextrin, having a structure corresponding to formula (2):

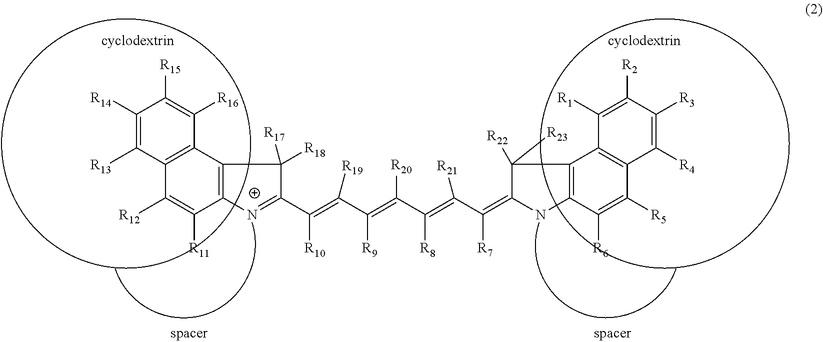

or a salt thereof, wherein R₁, R₂, R₃, R₄, R₅, R₆, R₇, R₁₀, R₁₁, R₁₂, R₁₃, R₁₄, R₁₅, R₁₆, R₁₇, R₁₈, R₁₉, R₂₀, R₂₁, R₂₂ and R₂₃ are each independently selected from the group consisting of hydrogen, alkyl, aryl, halogen, alkoxy, —NR^{a}R^{b}, —(NR^{a}R^{b}R^{c})⁺, carboxyl, formyl, sulfonyl, sulfonic acid, phosphate, alkyloxycarbonyl, aryloxycarbonyl, alkylcarbonyl, arylcarbonyl, and a heterocyclic ring; R₈ and R₉ are each independently selected from the group consisting of hydrogen, alkyl, aryl, halogen, alkoxyl, —NR^{a}R^{b}, —(NR^{a}R^{b}R^{c})⁺, carboxyl, formyl, sulfonyl, sulfonic acid, phosphate, alkyloxycarbonyl, aryloxycarbonyl, alkylcarbonyl, arylcarbonyl, and a heterocyclic ring, or R₈ and R₉ are taken together to form —CH₂—, —CH₂CH₂—, —CH₂CH₂CH₂— or —CH₂CH₂CH₂CH₂—; R^{a}, R^{b} and R^{c} are each independently selected from the group consisting of hydrogen and alkyl; and the alkyl group of R₁₋₂₃, R^{a}, R^{b} or W is optionally substituted with one or more substituents selected from the group consisting of alkyl, aryl, halogen, alkoxy, amino, carboxyl, formyl, sulfonyl, sulfonic acid, phosphate, alkyloxycarbonyl, aryloxycarbonyl, alkylcarbonyl, arylcarbonyl, and a heterocyclic ring. The fluorescence imaging agent may be a sodium salt, a potassium salt, or a magnesium salt of a chemical compound of formula (2). The fluorescence imaging agent may be a chloride salt of a chemical compound of formula (2).

The fluorescence imaging agent may be a cyclodextrin-bonded indocyanine compound in which an indocyanine is covalently bonded through an amide bond to a cyclic sugar chain cyclodextrin, represented by the chemical formula (1), wherein the compound is a cyclodextrin-bonded indocyanine compound represented by the following chemical formula (3):

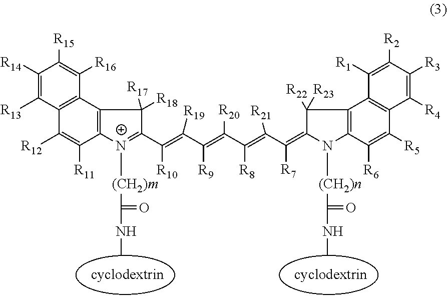

or a salt thereof, wherein R₁, R₂, R₃, R₄, R₅, R₆, R₇, R₁₀, R₁₁, R₁₂, R₁₃, R₁₄, R₁₅, R₁₆, R₁₇, R₁₈, R₁₉, R₂₀, R₂₁, R₂₂ and R₂₃ are each independently selected from the group consisting of hydrogen, alkyl, aryl, halogen, alkoxy, —NR^{a}R^{b}, —(NR^{a}R^{b}R^{c})⁺, carboxyl, formyl, sulfonyl, sulfonic acid, phosphate, alkyloxycarbonyl, aryloxycarbonyl, alkylcarbonyl, arylcarbonyl, and a heterocyclic ring; R₈ and R₉ are each independently selected from the group consisting of hydrogen, alkyl, aryl, halogen, alkoxyl, —NR^{a}R^{b}, —(NR^{a}R^{b}R^{c})⁺, carboxyl, formyl, sulfonyl, sulfonic acid, phosphate, alkyloxycarbonyl, aryloxycarbonyl, alkylcarbonyl, arylcarbonyl, and a heterocyclic ring, or R₈ and R₉ are taken together to form —CH₂—, —CH₂CH₂—, —CH₂CH₂CH₂— or —CH₂CH₂CH₂CH₂—; R^{a}, R^{b} and R^{c} are each independently selected from the group consisting of hydrogen and alkyl; and the alkyl group of R₁₋₂₃, R^{a}, R^{b} or R^{c} is optionally substituted with one or more substituents selected from the group consisting of alkyl, aryl, halogen, alkoxy, amino, carboxyl, formyl, sulfonyl, sulfonic acid, phosphate, alkyloxycarbonyl, aryloxycarbonyl, alkylcarbonyl, arylcarbonyl, and a heterocyclic ring; m and n are independently selected from an integer between 1 and 6. The fluorescence imaging agent may be a sodium salt, a potassium salt, or a magnesium salt of a chemical compound of formula (3). The fluorescence imaging agent may be a chloride salt of a chemical compound of formula (3).

The fluorescence imaging agent may be a cyclodextrin-bonded indocyanine compound in which an indocyanine is covalently bonded through an amide bond to a cyclic sugar chain cyclodextrin, represented by the chemical formula (2), wherein the compound is a cyclodextrin-bonded indocyanine compound represented by the following chemical formula (4):

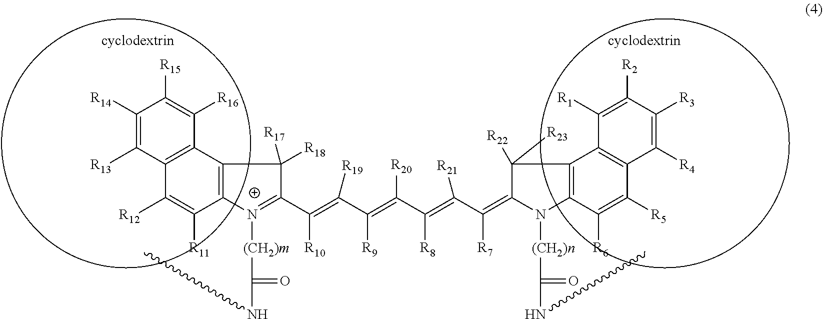

or a salt thereof, wherein R₁, R₂, R₃, R₄, R₅, R₆, R₇, R₁₀, R₁₁, R₁₂, R₁₃, R₁₄, R₁₅, R₁₆, R₁₇, R₁₈, R₁₉, R₂₀, R₂₁, R₂₂ and R₂₃ are each independently selected from the group consisting of hydrogen, alkyl, aryl, halogen, alkoxy, —NR^{a}R^{b}, —(NR^{a}R^{b}R^{c})⁺, carboxyl, formyl, sulfonyl, sulfonic acid, phosphate, alkyloxycarbonyl, aryloxycarbonyl, alkylcarbonyl, arylcarbonyl, and a heterocyclic ring; R₈ and R₉ are each independently selected from the group consisting of hydrogen, alkyl, aryl, halogen, alkoxyl, —NR^{a}R^{b}, —(NR^{a}R^{b}R^{c})⁺, carboxyl, formyl, sulfonyl, sulfonic acid, phosphate, alkyloxycarbonyl, aryloxycarbonyl, alkylcarbonyl, arylcarbonyl, and a heterocyclic ring, or R₈ and R₉ are taken together to form —CH₂—, —CH₂CH₂—, —CH₂CH₂CH₂— or —CH₂CH₂CH₂CH₂—; R^{a}, R^{b} and R^{c} are each independently selected from the group consisting of hydrogen and alkyl; and the alkyl group of R₁₋₂₃, R^{a}, R^{b} or W is optionally substituted with one or more substituents selected from the group consisting of alkyl, aryl, halogen, alkoxy, amino, carboxyl, formyl, sulfonyl, sulfonic acid, phosphate, alkyloxycarbonyl, aryloxycarbonyl, alkylcarbonyl, arylcarbonyl, and a heterocyclic ring; m and n are independently selected from an integer between 1 and 6. The fluorescence imaging agent may be a sodium salt, a potassium salt, or a magnesium salt of a chemical compound of formula (4). The fluorescence imaging agent may be a chloride salt of a chemical compound of formula (4).

The fluorescence imaging agent may be a cyclodextrin-bonded indocyanine compound represented by the chemical formula (3), which is a cyclodextrin-bonded indocyanine compound represented by the following chemical formula (5):

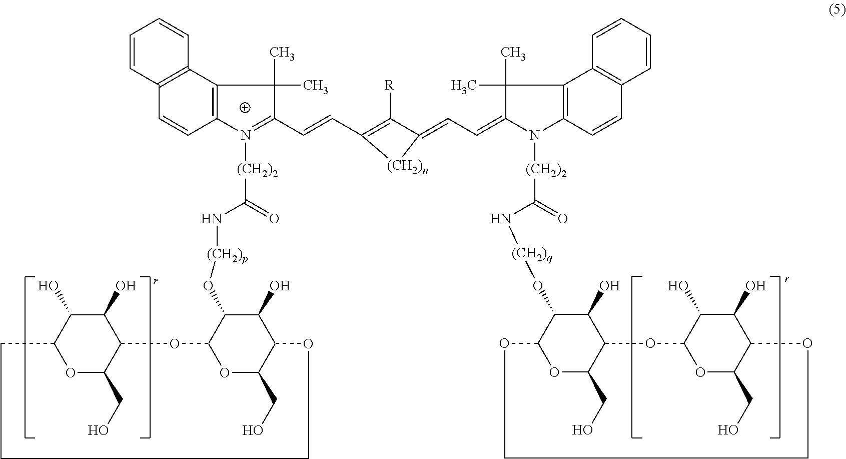

or a salt thereof, wherein m, n, p and q are each independently selected from an integer between 2 and 6; r is independently selected from an integer between 5 and 7; s is independently selected from an integer between 0 and 4; and R is selected from the group consisting of hydrogen, alkyl, aryl, halogen, alkoxy, amino, carboxyl, formyl, sulfonyl, sulfonic acid, alkyloxycarbonyl, aryloxycarbonyl, alkylcarbonyl, arylcarbonyl, and a heterocyclic ring.

The fluorescence imaging agent may be a cyclodextrin-bonded indocyanine compound represented by the chemical formula (4), which is a cyclodextrin-bonded indocyanine compound represented by the following chemical formula (6):

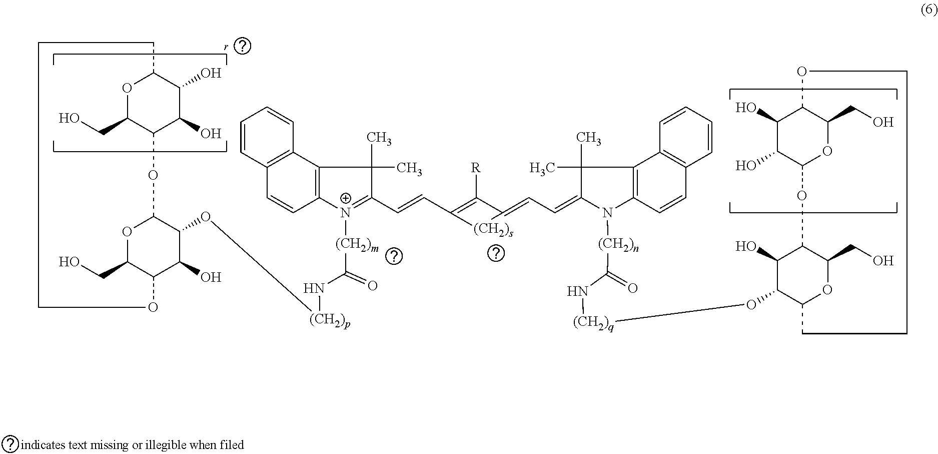

or a salt thereof, wherein m, n, p and q are each independently selected from an integer between 2 and 6; r is independently selected from an integer between 5 and 7; s is independently selected from an integer between 0 and 4; and R is selected from the group consisting of hydrogen, alkyl, aryl, halogen, alkoxy, amino, carboxyl, formyl, sulfonyl, sulfonic acid, alkyloxycarbonyl, aryloxycarbonyl, alkylcarbonyl, arylcarbonyl, and a heterocyclic ring.

The fluorescence imaging agent may be a cyclodextrin-bonded indocyanine compound represented by the chemical formula (3), which is a cyclodextrin-bonded indocyanine compound represented by the following chemical formula (7):

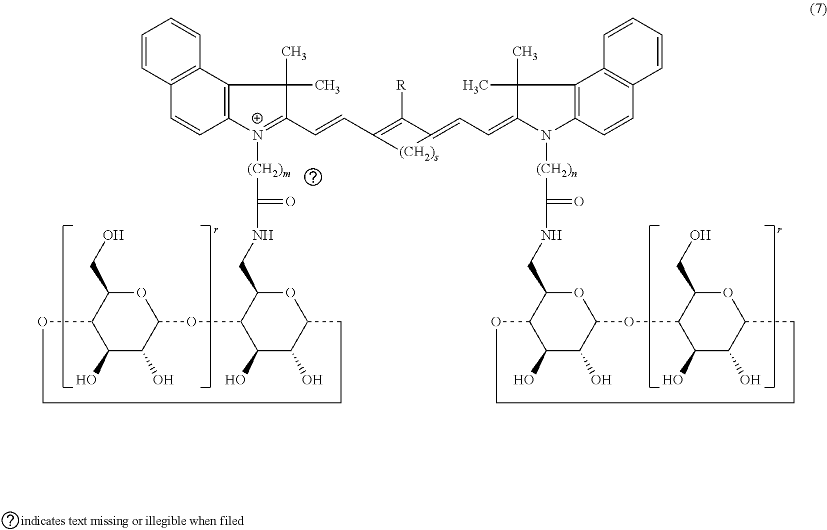

or a salt thereof, wherein m and n are independently selected from an integer between 2 and 6; r is independently selected from an integer between 5 and 7; s is independently selected from an integer between 0 and 4; and R is selected from the group consisting of hydrogen, alkyl, aryl, halogen, alkoxy, amino, carboxyl, formyl, sulfonyl, sulfonic acid, alkyloxycarbonyl, aryloxycarbonyl, alkylcarbonyl, arylcarbonyl, and a heterocyclic ring.

The fluorescence imaging agent may be a cyclodextrin-bonded indocyanine compound represented by the chemical formula (4) which is a cyclodextrin-bonded indocyanine compound represented by the following chemical formula (8):

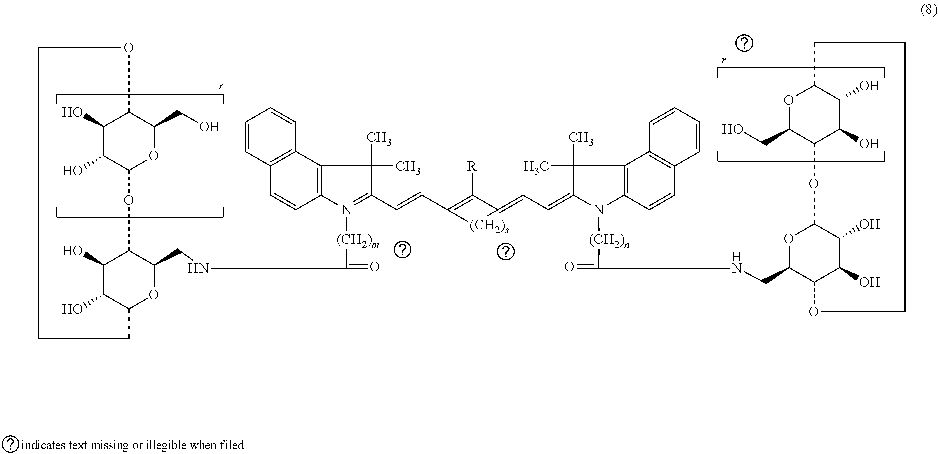

or a salt thereof, wherein m and n are independently selected from an integer between 2 and 6; r is independently selected from an integer between 5 and 7; s is independently selected from an integer between 0 and 4; and R is selected from the group consisting of hydrogen, alkyl, aryl, halogen, alkoxy, amino, carboxyl, formyl, sulfonyl, sulfonic acid, alkyloxycarbonyl, aryloxycarbonyl, alkylcarbonyl, arylcarbonyl, and a heterocyclic ring.

The fluorescence imaging agent may be a cyclodextrin-bonded indocyanine compound represented by the chemical formula (3), which is a cyclodextrin-bonded indocyanine compound represented by the following chemical formula (9):

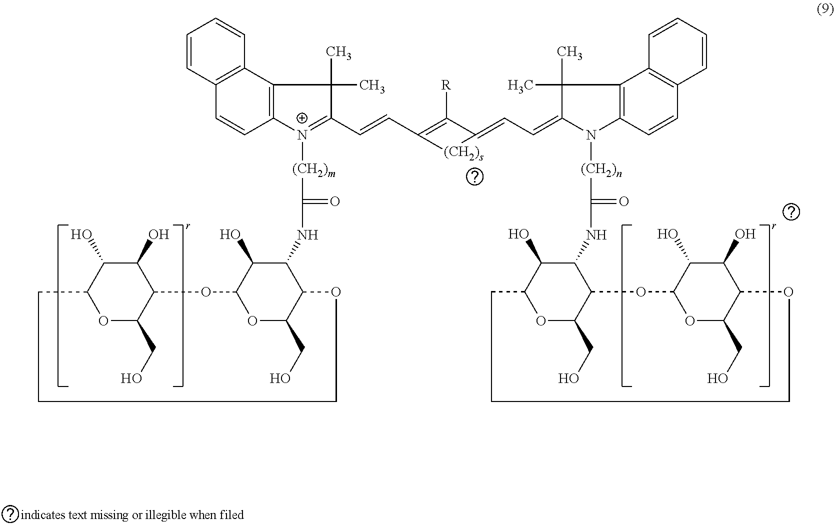

or a salt thereof, wherein m and n are independently selected from an integer between 2 and 6; r is independently selected from an integer between 5 and 7; s is independently selected from an integer between 0 and 4; and R is selected from the group consisting of hydrogen, alkyl, aryl, halogen, alkoxy, amino, carboxyl, formyl, sulfonyl, sulfonic acid, alkyloxycarbonyl, aryloxycarbonyl, alkylcarbonyl, arylcarbonyl, and a heterocyclic ring.

The fluorescence imaging agent may be a cyclodextrin-bonded indocyanine compound represented by the chemical formula (4), which is a cyclodextrin-bonded indocyanine compound represented by the following chemical formula (10):

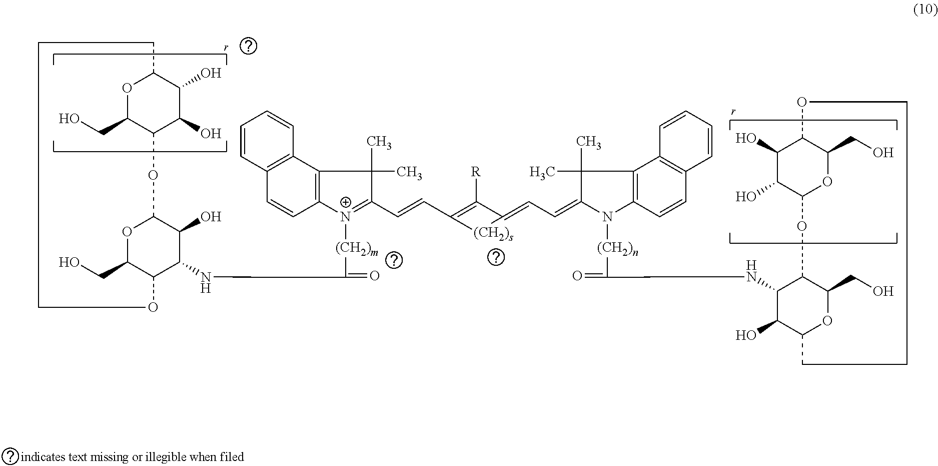

or a salt thereof, wherein m and n are independently selected from an integer between 2 and 6; r is independently selected from an integer between 5 and 7; s is independently selected from an integer between 0 and 4; and R is selected from the group consisting of hydrogen, alkyl, aryl, halogen, alkoxy, amino, carboxyl, formyl, sulfonyl, sulfonic acid, alkyloxycarbonyl, aryloxycarbonyl, alkylcarbonyl, arylcarbonyl, and a heterocyclic ring.

The fluorescence imaging agent may be a cyclodextrin-bonded indocyanine compound represented by the chemical formula (3), which is a cyclodextrin-bonded indocyanine compound represented by the following chemical formula (11):

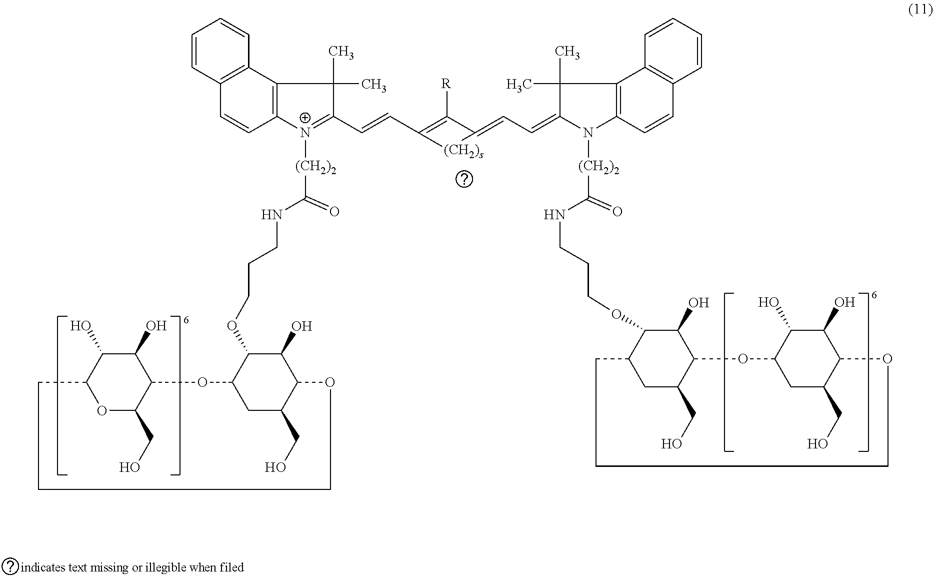

or a salt thereof, wherein s is independently selected from an integer between 0 and 4 and R is selected from the group consisting of hydrogen, alkyl, aryl, halogen, alkoxy, amino, carboxyl, formyl, sulfonyl, sulfonic acid, alkyloxycarbonyl, aryloxycarbonyl, alkylcarbonyl, arylcarbonyl, and a heterocyclic ring.

The fluorescence imaging agent may be a cyclodextrin-bonded indocyanine compound represented by the chemical formula (4), which is a cyclodextrin-bonded indocyanine compound represented by the following chemical formula (12):

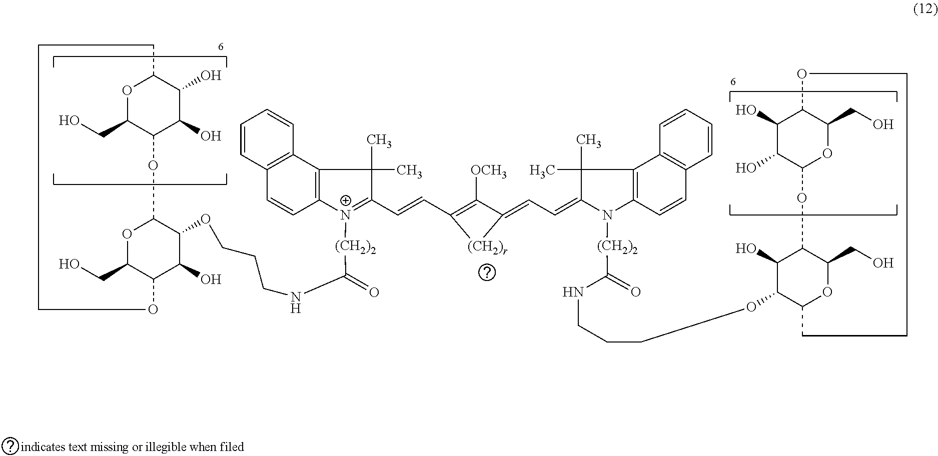

or a salt thereof, wherein s is independently selected from an integer between 0 and 4.

The fluorescence imaging agent may be a compound of formula (13):

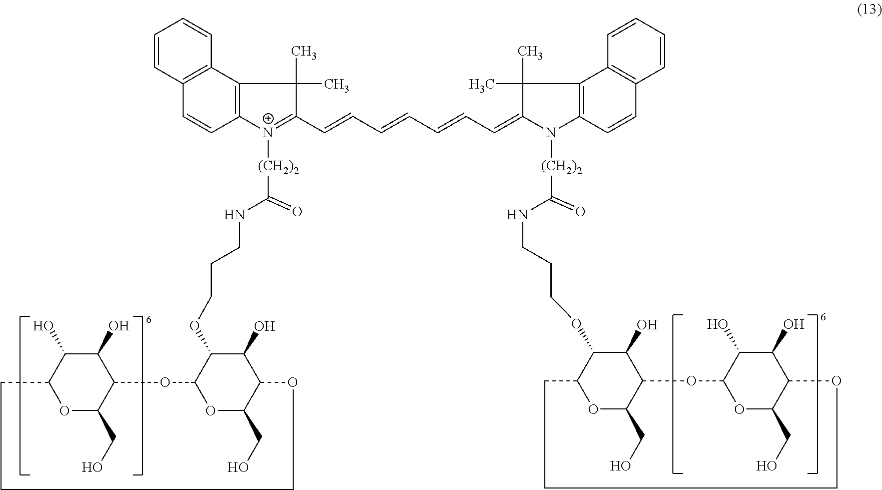

or a salt thereof.

The fluorescence imaging agent may be a cyclodextrin-bonded indocyanine compound in which at least a part of a naphthyl group of an indocyanine is included in a cavity of a cyclodextrin, having a structure corresponding to formula (14):

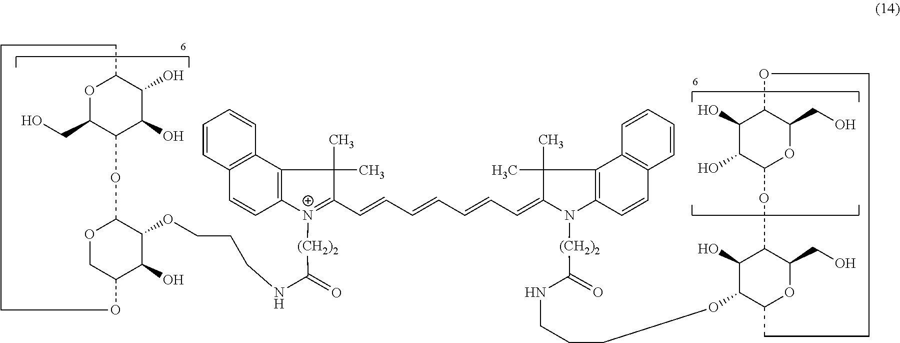

or a salt thereof.

The fluorescence imaging agent may be a compound of formula (15):

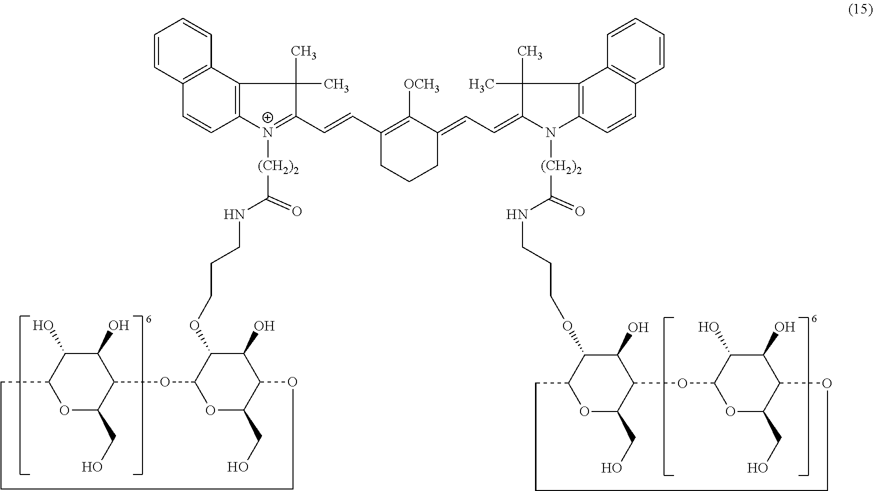

or a salt thereof.

The fluorescence imaging agent may be a cyclodextrin-bonded indocyanine compound in which at least a part of a naphthyl group of an indocyanine is included in a cavity of a cyclodextrin, having a structure corresponding to formula (16):

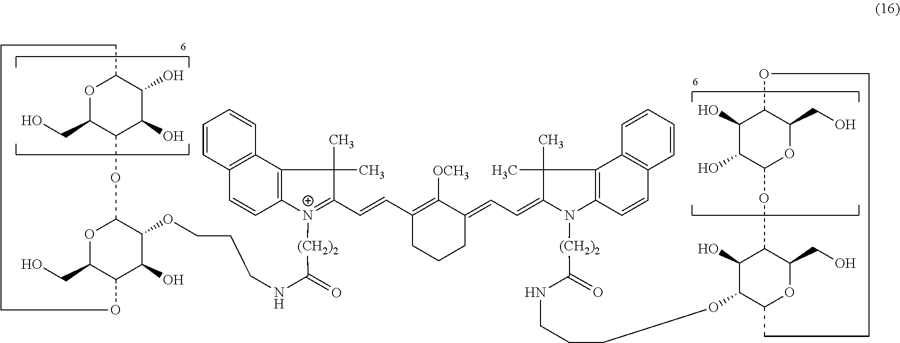

or a salt thereof.

The fluorescence imaging agent may be a compound of formula (17):

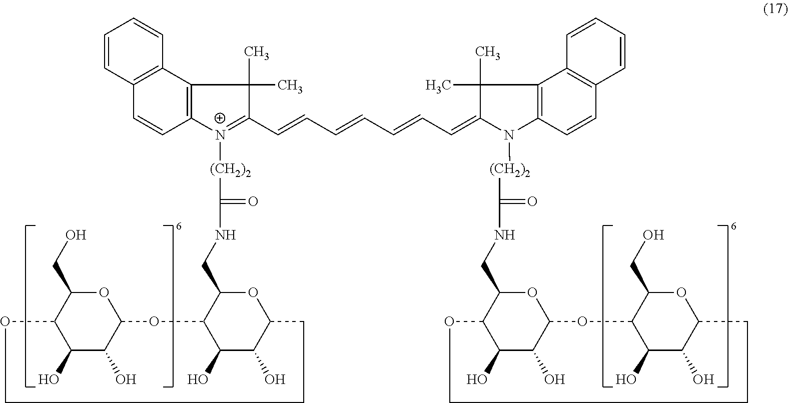

or a salt thereof.

The fluorescence imaging agent may be a compound of formula (18):

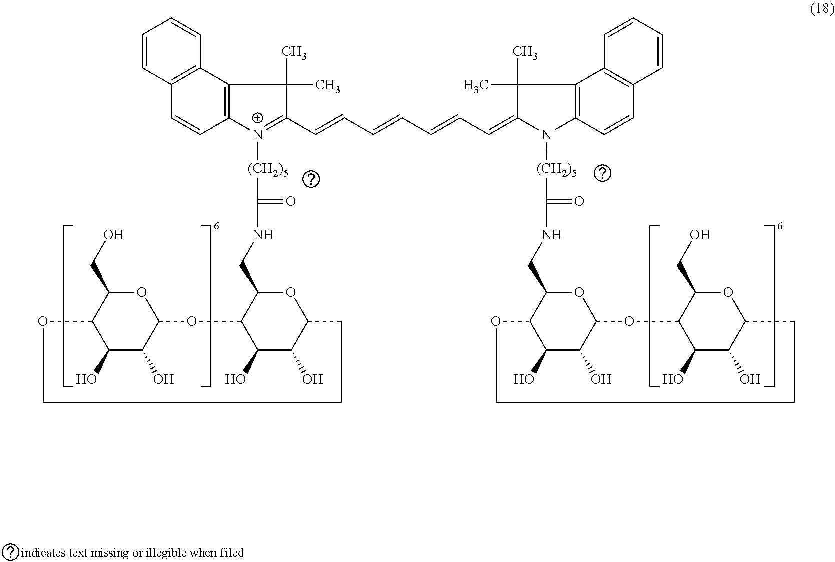

or a salt thereof.

The fluorescence imaging agent may be a compound of formula (19):

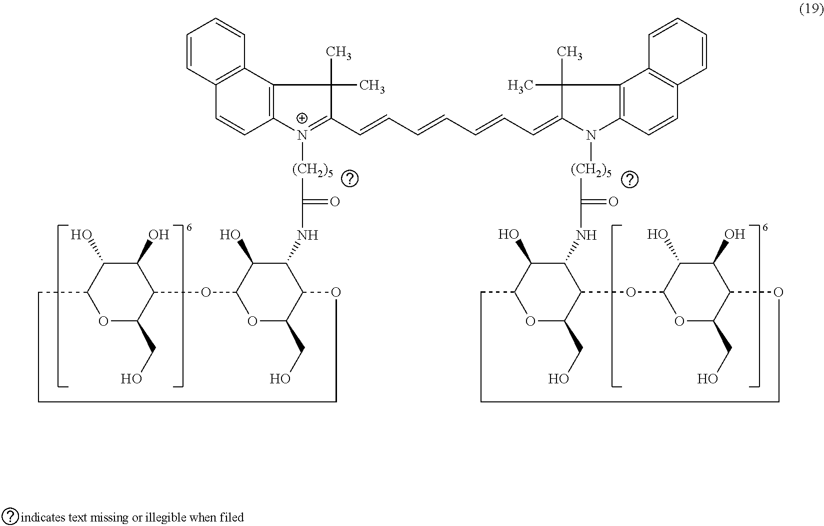

or a salt thereof.

The fluorescence imaging agent may be a compound of formula (20):

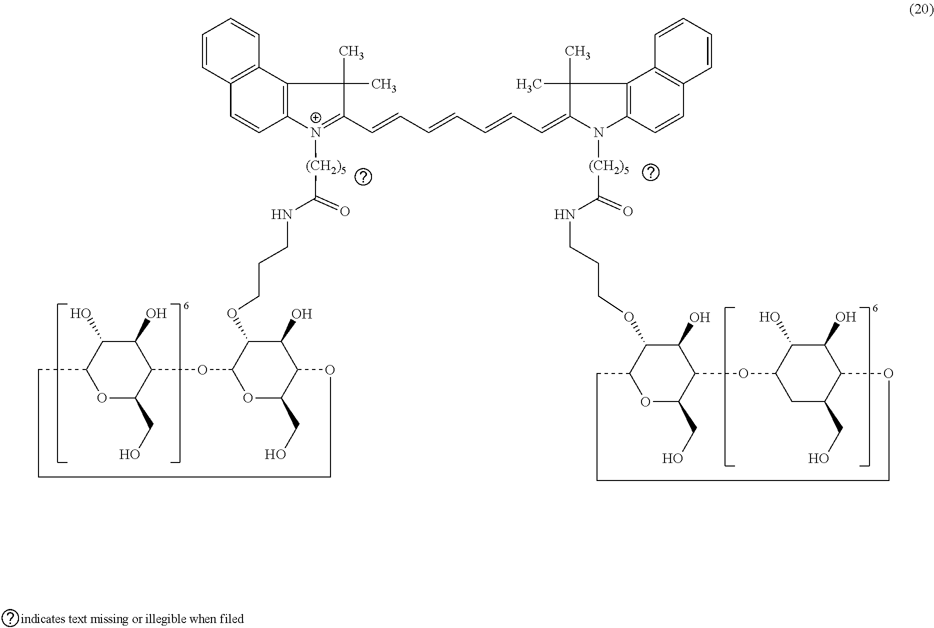

or a salt thereof.

In chemical formulae (1) to (20), “alkyl” refers to a linear or branched alkyl group having 1 to 20 carbon atoms, which is optionally substituted. For example, “alkyl” may include linear groups or branched groups including methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl and icosanyl.

A compound of any one of chemical formulae (1) to (20) may be administered at a dose of 0.3 mg, 1 mg, or 3 mg. A compound of any one of chemical formulae (1) to (20) may be administered at a dose of 0.1 mg to 10 mg, 0.1 mg to 9 mg, 0.1 mg to 8 mg, 0.1 mg to 7 mg, 0.1 mg to 6 mg, 0.1 mg to 5 mg, 0.1 mg to 4.5 mg, 0.1 mg to 4 mg, 0.1 mg to 3.5 mg, 0.1 mg to 3 mg, 0.1 mg to 2.5 mg, 0.1 mg to 2 mg, 0.1 mg to 1.5 mg, 0.1 mg to 1 mg, or 0.1 mg to 0.5 mg.

In chemical formulae (1) to (20), “aryl” refers to an aromatic hydrocarbon having 6 to 20 carbon atoms, such as phenyl and naphthyl.

In chemical formulae (1) to (20), “halogen” refers to F, Cl, Br, or I.

In chemical formulae (1) to (20), “heterocyclic ring” refers to a unsaturated, saturated, or partially saturated 5- to 7-membered monocyclic hetero ring group containing 1 to 4 heteroatoms selected from oxygen, sulfur, and nitrogen; or a bicyclic hetero ring group wherein a 5- to 7-membered monocyclic hetero ring is condensed with benzene or another 5- to 7-membered monocyclic hetero ring. For example, “heterocyclic ring” may include pyrrolyl, pyrazolyl, imidazolyl, triazolyl, tetrazolyl, pyridyl, pyrazinyl, pyrimidinyl, pyridazinyl, furyl, oxazolyl, isoxazolyl, oxadiazolyl, pyranyl, dioxolanyl, oxazinyl, oxadiazinyl, dioxazinyl, thienyl, thiazolyl, isothiazolyl, thiadiazolyl, thiopyranyl, thiazinyl,pyrrolidinyl, piperidyl, piperazinyl, morpholinyl, thiomorpholinyLindolyl, isoindolyl, indazolyl, benzimidazolyl, quinolyl, isoquinolyl, quinazolinyl, quinoxalinyl, naphthylidyl, cinnolinyl, phthalazinyl, indolizinyl, purinyl, quinolizyl, benzofuranyl, benzoxazolyl, benzisoxazolyl, benzoxadiazolyl, benzofurazanyl, benzothienyl, benzothiazolyl, benzisothiazolyl, benzothiadiazolyl, imidazopyridyl, imidazopyrazinyl, chromanyl, benzopyranyl or the like.

The salt of a compound of any one of chemical formulae (1) to (20) may be a pharmaceutically acceptable salt, and it may form a salt with acid or a salt with a base depending on the type of substituents. The salt of a compound of any one of chemical formulae (1) to (20) may be an acid addition salt. The salt may be a salt with an inorganic acid, such as hydrochloric acid, hydrobromic acid, hydriodic acid, sulfuric acid, nitric acid, or phosphoric acid. The salt may be a salt with an organic acid, such as formic acid, acetic acid, propionic acid, oxalic acid, malonic acid, succinic acid, fumaric acid, maleic acid, lactic acid, malic acid, mandelic acid, tartaric acid, dibenzoyl tartrate, ditoluoyl tartrate, citric acid, methanesulfonic acid, ethanesulfonic acid, benzenesulfonic acid, p-toluenesulfonic acid, aspartic acid, or glutamic acid. The salt of a compound of any one of chemical formulae (1) to (20) may be a base addition salt. The salt may be a salt with an inorganic base, such as sodium, potassium, magnesium, calcium, or aluminum. The salt may be a salt with an organic base, such as methylamine, ethylamine, ethanolamine, lysine, or ornithine. The salt may be a salt with various amino acids and amino acid derivatives such as acetylleucine, ammonium salts, and the like. The salt may be a sodium salt, a potassium salt, or a magnesium salt. The salt may be a halide salt. The salt may be a chloride salt.

Methods of preparing or using compounds of any of formulae (1)-(20) may be found, for example, in U.S. Pat. No. 10,086,090, the contents of which are hereby incorporated by reference in their entirety.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for assessing fluorescence imaging agent-based visual enhancement in a color image in which a fluorescence imaging agent signal is represented by a first color, the method comprising:
identifying at least one first region of the color image that is encompassed by a portion of anatomy or pathology of interest in which the fluorescence imaging agent is present;
identifying at least one second region of the color image that is not encompassed by the anatomy or pathology of interest; and
determining a measure of visual enhancement of the anatomy or pathology of interest in the color image by comparing a contribution of the first color relative to other colors in the at least one first region to a contribution of the first color relative to the other colors in the at least one second region.

2. The method of claim 1, wherein the measure of visual enhancement is associated with a first dose of the fluorescence imaging agent, and the method further comprises comparing the measure of visual enhancement associated with the first dose with a measure of visual enhancement associated with a second dose of the fluorescence imaging agent to determine a preferred dose of the fluorescence imaging agent for imaging the anatomy or pathology of interest.

3. The method of claim 1, wherein the color image was generated by a surgical visualization system, the measure of visual enhancement is associated with a first configuration of the surgical visualization system, and the method further comprises comparing the measure of visual enhancement associated with the first configuration of the surgical visualization system with a measure of visual enhancement associated with a second configuration of the surgical visualization system to determine a preferred configuration of the surgical visualization system for image-guided surgery of the anatomy or pathology of interest.

4. The method of claim 1, comprising determining the contribution of the first color relative to other colors in the at least one first region by calculating a first ratio of a measure of signal strength of the first color to a sum of measures of signal strengths of other colors in the at least one first region.

5. The method of claim 4, comprising determining the contribution of the first color relative to the other colors in the at least one second region by calculating a second ratio of a measure of signal strength of the first color to a sum of measures of signal strengths of other colors in the at least one second region, and wherein the measure of visual enhancement comprises a ratio of the first ratio to the second ratio.

6. The method of claim 1, further comprising comparing the measure of visual enhancement of the anatomy or pathology of interest in the color image with a measure of visual enhancement of the anatomy or pathology of interest in a second color image, wherein the second image does not include a contribution from the fluorescence imaging agent because the fluorescence imaging agent was not present in the at least a portion of anatomy or pathology of interest, the fluorescence imaging agent was not excited with fluorescence excitation light, or fluorescence imaging data was not used to generate the second image.

7. The method of claim 1, wherein identifying the at least one first region of the color image and the at least one second region of the color image comprises identifying a portion of the color image that includes the portion of the anatomy or pathology of interest and comparing pixel intensities of the first color within the portion of the color image to identify the at least one first region and the at least one second region.

8. The method of claim 1, wherein the anatomy or pathology of interest comprises a vessel.

9. The method of claim 8, wherein the vessel comprises a ureter.

10. The method of claim 1, wherein the fluorescence imaging agent is pudexacianinium chloride.

11. A method for assessing fluorescence imaging agent-based visual enhancement in a color image in which a fluorescence imaging agent signal is represented by a first color, the method comprising:
displaying the color image on a display to a user;
receiving, by a computing system, an input from the user corresponding to selection of at least one first region of the color image that is encompassed by a portion of anatomy or pathology of interest in which the fluorescence imaging agent is present;
receiving, by the computing system, an input from the user corresponding to selection of at least one second region of the color image that is not encompassed by the anatomy or pathology of interest; and
computing, by the computing system, a measure of visual enhancement of the anatomy or pathology of interest in the color image based on a contribution of the first color relative to other colors in the at least one first region to a contribution of the first color relative to the other colors in the at least one second region.

12. The method of claim 11, comprising:
illuminating tissue that comprises the anatomy or pathology of interest with fluorescence excitation light generated by an illumination system for causing fluorescence emission by the fluorescence imaging agent and with visible light generated by the illumination system;
detecting fluorescence emission and reflected light from the tissue by an image sensor assembly; and
generating the color image based on the light detected by the image sensor assembly.

13. The method of claim 11, wherein the measure of visual enhancement is associated with a first dose of the fluorescence imaging agent, and the method further comprises comparing the measure of visual enhancement associated with the first dose with a measure of visual enhancement associated with a second dose of the fluorescence imaging agent to determine a preferred dose of the fluorescence imaging agent for imaging the anatomy or pathology of interest.

14. The method of claim 11, wherein the color image was generated by a surgical visualization system, the measure of visual enhancement is associated with a first configuration of the surgical visualization system, and the method further comprises comparing the measure of visual enhancement associated with the first configuration of the surgical visualization system with a measure of visual enhancement associated with a second configuration of the surgical visualization system to determine a preferred configuration of the surgical visualization system for image-guided surgery of the anatomy or pathology of interest.

15. The method of claim 11, comprising computing the contribution of the first color relative to other colors in the at least one first region by calculating a first ratio of a measure of signal strength of the first color to a sum of measures of signal strengths of other colors in the at least one first region.

16. The method of claim 15, comprising computing the contribution of the first color relative to the other colors in the at least one second region by calculating a second ratio of a measure of signal strength of the first color to a sum of measures of signal strengths of other colors in the at least one second region, and wherein the measure of visual enhancement comprises a ratio of the first ratio to the second ratio.

17. The method of claim 11, wherein receiving inputs from the user corresponding to selections of the at least one first region of the color image and the at least one second region of the color image comprises receiving an input from the user corresponding to selection of a portion of the color image that includes the portion of the anatomy or pathology of interest and displaying to the user pixel intensities of the first color within the selected portion of the color image.

18. The method of claim 11, wherein the anatomy or pathology of interest comprises a vessel.

19. The method of claim 18, wherein the vessel comprises a ureter.

20. The method of claim 11, wherein the fluorescence imaging agent is pudexacianinium chloride.

21. A system for assessing fluorescence imaging agent-based visual enhancement in a color image in which a fluorescence imaging agent signal is represented by a first color, the system comprising:
an image guided surgery system comprising:
an illuminator configured to illuminate tissue that comprises the anatomy or pathology of interest with fluorescence excitation light generated for causing fluorescence emission by the fluorescence imaging agent and with visible light,
an image sensor assembly configured to detect fluorescence emission and reflected light from the tissue, and
an image processing system configured to generate the color image; and
a computing system comprising a display, one or more processors, memory, and one or more programs stored in the memory and including instruction for execution by the one or more processors for causing the computing system to:
display the color image on the display to a user,
receive an input from the user corresponding to selection of at least one first region of the color image that is encompassed by a portion of anatomy or pathology of interest in which the fluorescence imaging agent is present,
receive an input from the user corresponding to selection of at least one second region of the color image that is not encompassed by the anatomy or pathology of interest, and
compute a measure of visual enhancement of the anatomy or pathology of interest in the color image based on a contribution of the first color relative to other colors in the at least one first region to a contribution of the first color relative to the other colors in the at least one second region.

22. The system of claim 21, wherein the one or more programs include instructions for computing the contribution of the first color relative to other colors in the at least one first region by calculating a first ratio of a measure of signal strength of the first color to a sum of measures of signal strengths of other colors in the at least one first region.

23. The system of claim 22, wherein the one or more programs include instructions for computing the contribution of the first color relative to the other colors in the at least one second region by calculating a second ratio of a measure of signal strength of the first color to a sum of measures of signal strengths of other colors in the at least one second region, wherein the measure of visual enhancement comprises a ratio of the first ratio to the second ratio.

24. The system of claim 21, wherein receiving inputs from the user corresponding to selections of the at least one first region of the color image and the at least one second region of the color image comprises receiving an input from the user corresponding to selection of a portion of the color image that includes the portion of the anatomy or pathology of interest and displaying to the user pixel intensities of the first color within the selected portion of the color image. vessel.

25. The system of claim 21, wherein the anatomy or pathology of interest comprises a

26. The system of claim 25, wherein the vessel comprises a ureter.

27. The system of claim 21, wherein the fluorescence imaging agent is pudexacianinium chloride.

28. A method for determining a relationship between a fluorescence imaging agent-based visual enhancement in a color image of a patient and the concentration of the fluorescence imaging agent in a biological sample from the patient, wherein a fluorescence imaging agent signal in the color image is represented by a first color, the method comprising:
identifying at least one first region of the color image that is encompassed by a portion of anatomy or pathology of interest in which the fluorescence imaging agent is present;
identifying at least one second region of the color image that is not encompassed by the anatomy or pathology of interest;
determining a measure of visual enhancement of the anatomy or pathology of interest in the color image by comparing a contribution of the first color relative to other colors in the at least one first region to a contribution of the first color relative to the other colors in the at least one second region; and
calculating the relationship between the fluorescence imaging agent-based visual enhancement in the color image and the concentration of the fluorescence imaging agent in the biological sample.

29. The method of claim 28, wherein the measure of visual enhancement is determined at two or more different dosage amounts of the fluorescence imaging agent, and calculating the relationship comprises comparing the fluorescence imaging agent-based visual enhancement in the color image to the concentration of the fluorescence imaging agent in the biological sample at each of the two or more dosage amounts.

30. The method of claim 29, wherein determining the relationship comprises determining a dosage range over which there is a positive linear correlation between the fluorescence imaging agent-based visual enhancement in a color image of a patient and the concentration of the fluorescence imaging agent in a biological sample from the patient.

31. The method of claim 28, wherein the biological sample comprises urine, blood, lymphatic fluid, or feces.

32. A system for assessing fluorescent imaging agent-based visual enhancement in a color image, the system comprising:
one or more processors; and
memory communicatively coupled with the one or more processors, the memory storing instructions that, when executed by the one or more processors, cause the one or more processors to:
analyze pixel data of the color image to identify a first region of the color image associated with presence of a fluorescence imaging agent and a second region of the color image associated with lack of presence of the fluorescence imaging agent, the first region corresponding to a portion of anatomy or pathology of interest, and the fluorescence imaging agent being represented in a first color of the pixel data,
calculate a first contribution value for the first region based on pixel values of the first region that are associated with the first color and pixel values of the first region that are associated with at least one other color and a second contribution value for the second region based on pixel values of the second region that are associated with the first color and pixel values of the second region that are associated with the at least one other color, and
determine a visual enhancement value associated with the fluorescence imaging agent being present in the first region in the color image by comparing the first contribution value to the second contribution value.

33. The system of claim 32, wherein the visual enhancement value is included as part of a three-point or five-point visual enhancement scale indicating a visibility improvement of the first region of the anatomy or pathology of interest based on presence of the fluorescent imaging agent.

34. The system of claim 32, wherein the instructions, when executed by the one or more processors, further cause the one or more processors to identify the first region and the second region of the color image by:
comparing a pixel value for the first color of one or more first pixels in the first region to a first pixel color value threshold and a pixel value of the first color of one or more second pixels in the second region to the first pixel color value threshold, and
determining that the pixel value for the first color of one or more first pixels exceeds the first pixel color value threshold and that pixel value of the first color of the one or more second pixels is less than the first pixel color value threshold.

35. The system of claim 32, wherein the instructions, when executed by the one or more processors, further cause the one or more processors to combine fluorescent pixel data of a fluorescent image of the anatomy or pathology of interest with white light pixel data of a white light image of the anatomy or pathology of interest to generate the pixel data of the color image.

36. The system of claim 32, wherein the measure of visual enhancement comprises a measure of the perceived conspicuity of the anatomy or pathology of interest.
